SEQUENCE LISTING (45 sequences)

SEQ ID NO: 1 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 10 aa, synthetic construct
  REGION 1..10: note = CDR1H of the 12E12 antibody
GFTFSDYYMY

SEQ ID NO: 4 - protein, 17 aa, synthetic construct
  REGION 1..17: note = CDR2H of the 12E12 antibody
YINSGGGSTYYPDTVKG

SEQ ID NO: 5 - protein, 10 aa, synthetic construct
  REGION 1..10: note = CDR3H of the 12E12 antibody
RGLPFHAMDY

SEQ ID NO: 6 - protein, 11 aa, synthetic construct
  REGION 1..11: note = CDR1L of the 12E12 antibody
SASQGISNYLN

SEQ ID NO: 7 - protein, 7 aa, synthetic construct
  REGION 1..7: note = CDR2L of the 12E12 antibody
YTSILHS

SEQ ID NO: 8 - protein, 9 aa, synthetic construct
  REGION 1..9: note = CDR3L of the 12E12 antibody
QQFNKLPPT

SEQ ID NO: 9 - protein, 10 aa, synthetic construct
  REGION 1..10: note = CDR1H of the 11B6 antibody
GYSFTGYYMH

SEQ ID NO: 10 - protein, 17 aa, synthetic construct
  REGION 1..17: note = CDR2H of the 11B6 antibody
RINPYNGATSYNQNFKD

SEQ ID NO: 11 - protein, 5 aa, synthetic construct
  REGION 1..5: note = CDR3H of the 11B6 antibody
EDYVY

SEQ ID NO: 12 - protein, 16 aa, synthetic construct
  REGION 1..16: note = CDR1L of the 11B6 antibody
RSSQSLVHSNGNTYLH

SEQ ID NO: 13 - protein, 7 aa, synthetic construct
  REGION 1..7: note = CDR2L of the 11B6 antibody
KVSNRFS

SEQ ID NO: 14 - protein, 9 aa, synthetic construct
  REGION 1..9: note = CDR3L of the 11B6 antibody
SQSTHVPWT

SEQ ID NO: 15 - protein, 10 aa, synthetic construct
  REGION 1..10: note = CDR1H of the 12B4 antibody
GYTFTDYVLH

SEQ ID NO: 16 - protein, 17 aa, synthetic construct
  REGION 1..17: note = CDR2H of the 12B4 antibody
YINPYNDGTKYNEKFKG

SEQ ID NO: 17 - protein, 12 aa, synthetic construct
  REGION 1..12: note = CDR3H of the 12B4 antibody
GYPAYSGYAMDY

SEQ ID NO: 18 - protein, 11 aa, synthetic construct
  REGION 1..11: note = CDR1L of the 12B4 antibody
RASQDISNYLN

SEQ ID NO: 19 - protein, 7 aa, synthetic construct
  REGION 1..7: note = CDR2L of the 12B4 antibody
YTSRLHS

SEQ ID NO: 20 - protein, 9 aa, synthetic construct
  REGION 1..8: note = CDR3L of the 12B4 antibody
HHGNTLPWT

SEQ ID NO: 21 - protein, 116 aa, synthetic construct
  REGION 1..116: note = Amino acid sequence of variable heavy chain region (VH) (v2) ofHumanized 11B6
EVQLVQSGAEVKKPGASVKISCKASGYSFTGYYMHWVKQAHGQGLEWIGRINPYNGATSYNQNFKDRATLTVDKSTSTAYMELSSLRSEDTAVYYCAREDYVYWGQGTTVTVSSAS

SEQ ID NO: 22 - protein, 108 aa, synthetic construct
  REGION 1..108: note = Amino acid sequence of variable light chain (VL) Vk (v2) ofhumanized 11B6 VL
DVVMTQSPLSLPVTLGQPASISCRSSQSLVHSNGNTYLHWYQQRPGQSPRLLIYKVSNRFSGVPDRFSGSGSGTDFTLKISRVEAEDVGVYFCSQSTHVPWTFGGGTK

SEQ ID NO: 23 - protein, 116 aa, synthetic construct
  REGION 1..116: note = Amino acid sequence of variable heavy chain region VH (v3) ofhumanized 11B6
EVQLVQSGAEVKKPGASVKVSCKASGYSFTGYYMHWVRQAPGQGLEWIGRINPYNGATSYNQNFKDRVTLTVDKSTSTAYMELSSLRSEDTAVYYCAREDYVYWGQGTTVTVSSAS

SEQ ID NO: 24 - protein, 123 aa, synthetic construct
  REGION 1..123: note = VH amino acid sequence of mAb3 (12B4)
EVQLQQSGPELVKPGASVKMSCKASGYTFTDYVLHWVKQKPGQGLEWIGYINPYNDGTKYNEKFKGKATLTSDKSSSTAYMELSSLTSEDSAVYYCARGYPAYSGYAMDYWGQGTSVTVSSAS

SEQ ID NO: 25 - protein, 103 aa, synthetic construct
  REGION 1..103: note = VL amino acid sequence of mAb3 (12B4)
DIQMTQTTSSLSASLGDRVTISCRASQDISNYLNWYQQKPDGTVKLLIYYTSRLHSGVPSRFSGSGSGTDYSLTISNLEQEDIATYFCHHGNTLPWTFGGGTK

SEQ ID NO: 26 - protein, 121 aa, synthetic construct
  REGION 1..121: note = VH amino acid sequence of mAb4 (24A3 HC)
DVQLQESGPDLVKPSQSLSLTCTVTGYSITSDYSWHWIRQFPGNKLEWMGYIYYSGSTNYNPSLKSRISITRDTSKNQFFLQLNSVTTEDSATYFCARFYYGYSFFDYWGQGTTLTVSSAS

SEQ ID NO: 27 - protein, 102 aa, synthetic construct
  REGION 1..102: note = VL amino acid sequence of mAb4 (24A3 KC)
QIVLTQSPAFMSASPGEKVTMTCSASSSVSYMHWYQQKSGTSPKRWIYDTSKLASGVPARFSGSGSGTSYSLTISSMEAEDAATYYCQQWSSNPLTFGAGTK

SEQ ID NO: 28 - protein, 128 aa, synthetic construct
  REGION 1..128: note = VH amino acid sequence of mAb5
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSAS

SEQ ID NO: 29 - protein, 103 aa, synthetic construct
  REGION 1..103: note = VL amino acid sequence of mAb5
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTK

SEQ ID NO: 30 - protein, 121 aa, synthetic construct
  REGION 1..121: note = VH amino acid sequence of mAb6 (12E12 H3 Humanized HC)
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSAS

SEQ ID NO: 31 - protein, 103 aa, synthetic construct
  REGION 1..103: note = VL amino acid sequence of mAb6 (Humanized K2 12E12)
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTK

SEQ ID NO: 32 - protein, 81 aa, synthetic construct
  REGION 1..81: note = Amino acid sequence of variable heavy chain region (VH) of 15B10
SVKMSCKASGYTFTDYVISWVKQRTGQGLEWIGDIYPGSGYSFYNENFKGKATLTADKSSTTAYMQLSSLTSEDSAVYFCA

SEQ ID NO: 33 - protein, 76 aa, synthetic construct
  REGION 1..76: note = Amino acid sequence of variable light chain (VL) 15B10
ASISCRSSQSLVHSNGNTYLHWYLQKPGQSPKLLIYKVSNRFSGVPDRFSGSGSGTNFTLKISRVEAEDLGLYFCS

SEQ ID NO: 34 - protein, 82 aa, synthetic construct
  REGION 1..82: note = Amino acid sequence of variable heavy chain region (VH) of 2G3
SSVKMSCKASGYTFTDYVISWVKQRTGQGLEWIGDIYPGSGYSFYNENFKGKATLTADKSSTTAYMQLSSLTSEDSAVYFCA

SEQ ID NO: 35 - protein, 74 aa, synthetic construct
  REGION 1..74: note = Amino acid sequence of variable light chain (VL) 2G3
VTLTCRSSTGAVTTSNYANWVQEKPDHLFTGLIGGTNNRVSGVPARFSGSLIGDKAALTITGAQTEDEAIYFCA

SEQ ID NO: 36 - protein, 456 aa, synthetic construct
  REGION 1..456: note = Amino acid sequence of the heavy chain of 4C7
QVQLQQSGAELVRPGASVTLSCKASGYTFIDHDMHWVQQTPVYGLEWIGAIDPETGDTGYNQKFKGKAILTADKSSRTAYMELRSLTSEDSAVYYCTIPFYYSNYSPFAYWGQGALVTVSAAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCADLLGGPSVFIFPPKIKDVLMISLSPMVTCVVVDVSEDDPDAQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKAS

SEQ ID NO: 37 - protein, 213 aa, synthetic construct
  REGION 1..213: note = Amino acid sequence of light chain of 4C7
QIVLSQSPAILSASPGEKVTMTCRASSSVSYMHWYQRKPGSSPKPWIYATSNLASGVPARFSGSGSGTSYSLTISRVEAEDAATYYCQQWSSNPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 38 - protein, 27 aa, synthetic construct
  REGION 1..27: note = FlexV1
QTPTNTISVTPTNNSTPTNNSNPKPNP

SEQ ID NO: 39 - protein, 25 aa, synthetic construct
  REGION 1..25: note = f1 linker
SSVSPTTSVHPTPTSVPPTPTKSSP

SEQ ID NO: 40 - protein, 25 aa, synthetic construct
  REGION 1..25: note = f2 linker
PTSTPADSSTITPTATPTATPTIKG

SEQ ID NO: 41 - protein, 25 aa, synthetic construct
  REGION 1..25: note = f3 linker
TVTPTATATPSAIVTTITPTATTKP

SEQ ID NO: 42 - protein, 25 aa, synthetic construct
  REGION 1..25: note = f4 linker
TNGSITVAATAPTVTPTVNATPSAA

SEQ ID NO: 43 - protein, 439 aa, synthetic construct
  REGION 1..439: note = hAnti-CD40VK2-LV-hIgGK-C-ViralSARS-CoV-2-Spike-RBDC 221S SAD-K var
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVKGFNCYFPLQSYGFQPTYGVGYQPYRVVVLSFELLHAPATVCGPKKSTNLVKNKSVNF

SEQ ID NO: 44 - protein, 439 aa, synthetic construct
  REGION 1..439: note = hAnti-CD40VK2-LV-hIgGK-C-ViralSARS-CoV-2-Spike-RBDC 221S SAD-K var
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVQGFNCYFPLQSYGFQPTYGVGYQPYRVVVLSFELLHAPATVCGPKKSTNLVKNKSVNF

SEQ ID NO: 45 - protein, 611 aa, synthetic construct
  REGION 1..611: note = hAnti-CD40VH3-LV-hIgG4H-C-f4-ViralSARS-Cov-2-Npep2
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASTNGSITVAATAPTVTPTVNATPSAAASRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSM

(19) United States
(12) Patent Application Publication
(10) Pub. No.: US 2025/0025548 A1
(43) Pub. Date: Jan. 23, 2025

(54) UNIVERSAL SARBECOVIRUS VACCINES

(71) Applicants: Institut National de la Santé et de la Recherche Médicale, Paris (FR); Assistance Publique - Hopitaux de Paris, Paris (FR); Baylor Research Institute, Dallas, TX (US); Universite Paris Est Creteil Val De Marne, Creteil (FR)

(72) Inventors: Yves LEVY, Creteil Cedex (FR); Gérard ZURAWSKI, Dallas, TX (US); Sandra ZURAWSKI, Dallas, TX (US); Mireille CENTLIVRE, Creteil Cedex (FR); Christine LACABARATZ, Creteil Cedex (FR); Sylvain CARDINAUD, Creteil Cedex (FR); Mathieu SURENAUD, Creteil Cedex (FR)

(73) Assignees: Institut National de la Santé et de la Recherche Médicale, Paris (FR); Assistance Publique - Hopitaux de Paris, Paris (FR); Baylor Research Institute, Dallas, TX (US); Universite Paris Est Creteil Val De Marne, Creteil (FR)

(21) Appl. No.: 18/710,734

(22) PCT Filed: Nov. 16, 2022
(86) PCT No.: PCT/EP2022/082134

§ 371 Date: May 16, 2024

(30) Foreign Application Priority Data
Nov. 17, 2021 (EP) 21306597.2

(51) Int. Cl. A61K 39/215; A61P 31/14; C07K 16/28; C12N 7/00

(52) U.S. Cl. CPC A61K 39/215; A61P 31/14; C07K 16/2851; C07K 16/2878; C12N 7/00; C07K 2317/24; C07K 2317/565; C12N 2770/20034

(57) ABSTRACT

Sarbecoviruses (lineage B of genus Betacoronavirus) have caused two major outbreaks during the past two decades: SARS-CoV in 2003, and SARS-CoV-2 from 2019. SARS-CoV-2 vaccines will be essential to reduce morbidity and mortality. Therefore, universal vaccines against this class of viruses are key to ending the current pandemic, but also for preventing additional emergent variations and future outbreaks of SARS-like viruses that are continuously found from nature reservoirs. Now, the inventors produced an antibody that is directed against a surface antigen (i.e., CD40) of an antigen presenting cell (i.e., dendritic cell) wherein the heavy chain is conjugated or fused to a protein N polypeptide of SAR-CoV-2 and the light chain is fused to a RBD polypeptide. In particular, the inventors show that said antibody could elicit immune responses against Sarbecoviruses.

### FIELD OF THE INVENTION

The present invention is in the field of medicine, in particular virology.

### BACKGROUND OF THE INVENTION

Sarbecoviruses (lineage B of genus Betacoronavirus) have caused two major outbreaks during the past two decades: SARS-CoV in 2003, and SARS-CoV-2 from 2019. Coronavirus-induced disease 2019 (COVID-19) is indeed caused by a zoonotic virus, severe acute respiratory syndrome coronavirus 2 (SARS-CoV-2), which has rapidly spread during 2020 and a half, infecting over 100 million humans and causing more than two million deaths worldwide. Durable control of the pandemic requires mass vaccination strategies, for which the first vaccine candidates became available at the end of 2020. Although there are a limited number of previously licensed vector-based vaccines for human use, recombinant DNA vector and synthetic mRNA vaccines have nevertheless become the most advanced in the fight against COVID-19 because of the many possibilities offered for genetic engineering and rapid scalability^{1,2,3,4}. Given that the benefits outweigh the risks for their use in humans, several vaccines, including mRNA-derived, vector-based vaccines and virus inactivated vaccines have been authorized for an emergency use to fight the spread of the disease in humans^{3,5,6,7,8,9,10,11}. More recently, a new generation of subunit vaccines targeting viral antigens to CD40-expressing antigen-presenting cells was reported¹². The receptor-binding domain (RBD) of SARS-CoV-2 spike is the major immunogen for current vaccines. However, coronaviral spikes show considerable genetic diversity among different species and variants. Moreover, convalescent COVID-19 patients show little cross-neutralizing activity against SARS-CoV-2 suggesting that the current immunogen may not elicit broad protection against Sarbecoviruses. Therefore, universal vaccines against this class of viruses are key to ending the current pandemic, but also for preventing additional emergent variations and future outbreaks of SARS-like viruses that are continuously found from nature reservoirs.

### SUMMARY OF THE INVENTION

The present invention is defined by the claims. In particular, the present invention relates to an antibody that is directed against a surface antigen of an antigen presenting cell wherein the heavy chain is conjugated or fused to the Npep2 polypeptide, and the light chain is conjugated or fused to
the RBD polypeptide that comprises the K417N, L452R, T478K, E484K, and N501Y naturally occurring mutations and that comprises the C538S non naturally occurring mutation
or the RBD polypeptide that comprises the K417N, L452R, T478K, E484Q and N501Y naturally occurring mutations and that comprises the C538S non naturally occurring mutation.

The present invention also related to the use of said antibody as a universal Sarbecovirus vaccine.

### DETAILED DESCRIPTION OF THE INVENTION

### Definitions

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase “nucleotide sequence that encodes a protein or a RNA” may also include introns to the extent that the nucleotide sequence encoding the protein may in some version contain an intron(s).

As used herein, the expression “derived from” refers to a process whereby a first component (e.g., a first polypeptide), or information from that first component, is used to isolate, derive or make a different second component (e.g., a second polypeptide that is different from the first one).

As used herein, the “percent identity” between the two sequences is a function of the number of identical positions shared by the sequences (i.e., % identity=number of identical positions/total number of positions ×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “coronavirus” has its general meaning in the art and refers to any member of members of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. These coronaviruses infect a variety of mammals and birds. They cause respiratory infections (common), enteric infections (mostly in infants >12 mo.), and possibly neurological syndromes. Coronaviruses are transmitted by aerosols of respiratory secretions.

As used herein, the term “Betacoronavirus”, also known as β-CoVs or Beta-CoVs has its general meaning in the art and refers to one of four genera (Alpha-, Beta-, Gamma-, and Delta-) of coronaviruses. The betacoronavirus genus comprises four lineages: A, B, C, D.

As used herein, the term “Sarbecovirus” has its general meaning in the art and refers to the lineage B of Betacoronavirus. The Sarbecoviruses of the greatest clinical importance concerning humans are Severe acute respiratory syndrome coronavirus (SARS-CoV or SARS-CoV-1) and Severe acute respiratory syndrome coronavirus 2 (SARS-CoV-2). The subgenus also includes but is not limited to Bat SC2r-CoV RaTG13, Bat SC2r-CoV RacCS203, Pangolin SC2r-CoV GX-P4L, Pangolin SC2r-CoV GX-P5L, Bat SC2r-CoV ZC45, Bat SC2r-CoV ZXC21, Bat SC2r-CoV Rc-o319, Bat SC1r-CoV WIV1, Bat CoV WIV16, Bat CoV Rs3367, Bat CoV LYRa11, Bat SC1r-CoV Cp/Yunnan2011, Bat CoV Rs-YN2018B, Bat CoV Rs7327, Bat CoV RsSHC014, Bat CoV Rs4231, Bat CoV Rs4084, Bat CoV Rs4081, Bat CoV Rs672, Bat CoV Rs4237, Bat SC1r-CoV YNLF_31C, Bat SC1r-CoV Rp3, BtR1-BetaCoV/SC2018, Bat SC1r-CoV Rf1, Bat SC1r-CoV HeB2013, Bat SC1r-CoV Rp/Shaanxi2011, Bat SC1r-CoV Rm1, Bat SC1r-CoV HuB2013, Bat SC1r-CoV HKU3-1, Bat SC1r-CoV Longquan-140, Bat SC1r-CoV BM48-31/BGR/2008, and Bat SC1r-CoV BtKY72.

As used herein, the term “Severe Acute Respiratory Syndrome coronavirus 2” or “SARS-Cov-2” has its general meaning in the art and refers to the strain of coronavirus that causes coronavirus disease 2019 (COVID-19), a respiratory syndrome that manifests a clinical pathology resembling mild upper respiratory tract disease (common cold-like symptoms) and occasionally severe lower respiratory tract illness and extra-pulmonary manifestations leading to multi-organ failure and death.

As used herein, the term “nucleoprotein” or “protein N” refers to the SARS-CoV-2 protein that packages the positive strand viral genome RNA into a helical ribonucleocapsid (RNP) and plays a fundamental role during virion assembly through its interactions with the viral genome and membrane protein M. Typically, the nucleoprotein has the amino acid sequence as set forth in SEQ ID NO:1.

>sp|P0DTC9|NCAP_SARS2 Nucleoprotein OS = Severe
acute respiratory syndrom coronavirus 2 OX =
2697049 GN = N PE = 1 SV = 1. The polypeptides
Npep2 is underlined.
SEQ ID NO: 1
MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDFSKQLQQSMSSADSTQA

As used herein, the term “Npep2 polypeptide” refers to the SARS-CoV-2 polypeptide that derives from the protein N of SAR-CoV-2 and that consists of the amino acid sequence that ranges from the residue at position 276 to the residue at position 411 in SEQ ID NO:1.

As used herein, the term “spike protein” or “protein S” refers to the SARS-CoV-2 spike glycoprotein that binds its cellular receptor (i.e., ACE2), and mediates membrane fusion and virus entry. Each monomer of trimeric S protein is about 180 kDa, and contains two subunits, S1 and S2, mediating attachment and membrane fusion, respectively. In particular, Spike protein S1 attaches the virion to the cell membrane by interacting with host receptor (i.e., human ACE2 receptor) via its “receptor-binding domain” also named “RBD.” Spike protein S2 mediates fusion of the virion and cellular membranes by acting as a class I viral fusion protein. Under the current model, the protein has at least three conformational states: pre-fusion native state, pre-hairpin intermediate state, and post-fusion hairpin state. During viral and target cell membrane fusion, the coiled coil regions (heptad repeats) assume a trimer-of-hairpins structure, positioning the fusion peptide in close proximity to the C-terminal region of the ectodomain. The formation of this structure appears to drive apposition and subsequent fusion of viral and target cell membranes. Spike protein S2′ acts as a viral fusion peptide which is unmasked following S2 cleavage occurring upon virus endocytosis. Typically, the spike protein has the amino acid sequence as set forth in SEQ ID NO:2.

>sp|PODTC2|SPIKE_SARS2 Spike glycoprotein OS =
Severe acute respiratory syndrome coronavirus 2
OX = 2697049 GN = SPE = 1 SV = 1. The RBD is
underlined in the sequence.
SEQ ID NO: 2
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT

As used herein the term “RBD polypeptide” refers to the polypeptide that consists of the amino acid sequence that ranges from the amino acid residue at position 319 to the amino acid residue at position 541 in SEQ ID NO:2.

As used herein, the term “mutation” has its general meaning in the art and refers to a substitution, deletion or insertion. In particular, the term “substitution” means that a specific amino acid residue at a specific position is removed and another amino acid residue is inserted into the same position. Within the specification, the mutation are references according to the standard mutation nomenclature. In particular the term “mutation” encompasses “naturally-occurring mutations” and “non-naturally occurring mutations”.

As used herein, the term “naturally occurring mutation” refers to any mutation that can be found in the naturally occurring variants of the SARS-CoV-2 polypeptides and that typically include the B.1.1.7 lineage (a.k.a. 20I/501Y.V1 Variant of Concern (VOC) 202012/01 or Alpha VOC), the B.1.351 lineage (a.k.a. 20H/501Y.V2 or Beta VOC) and the P.1 lineage (a.k.a. 20J/501Y.V3 or Gamma VOC) and the B.1.617.2 lineage (a.k.a. Delta VOC). Said mutation are well-known in the art and include those described in the following references that are incorporated by reference:
(1) Jie Hu et al. The D614G mutation of SARS-CoV-2 spike protein enhances viral infectivity and decreases neutralization sensitivity to individual convalescent sera. bioRxviv (2020).
(2) Korber B. et al. Spike mutation pipeline reveals the emergence of a more transmissible form of SARS-CoV-2. bioRxviv (2020). doi.org/10.1101/2020.04.29.069054.
(3) Lizhou Zhang et al. The D614G mutation in the SARS-CoV-2 spike protein reduces S1 shedding and increases infectivity. bioRxviv (2020). doi.org/10.1101/2020.06.12.148726.
(4) Junxian Ou et al. Emergence of RBD mutations in circulating SARS-CoV-2 strains enhancing the structural stability and human ACE2 receptor affinity of the spike protein. bioRxiv (2020). doi:10.1101/2020.03.15.991844v4
(5) Saha, P. et al. Mutations in Spike Protein of SARS-CoV-2 Modulate Receptor Binding, Membrane Fusion and Immunogenicity: An Insight into Viral Tropism and Pathogenesis of COVID-19. chemRxiv (2020). doi:10.26434/chemrxiv.12320567.v1
(6) Jian Shang, Yushun Wan, Chuming Luo, Gang Ye, Qibin Geng, Ashley Auerbach, Fang Li. Cell entry mechanisms of SARS-CoV-2. Proceedings of the National Academy of Sciences May 2020, 117 (21) 11727-11734; DOI: 10.1073/pnas.2003138117
(7) Allison J. Greaney, Andrea N. Loes, Katharine H. D. Crawford, Tyler N. Starr, Keara D. Malone, Helen Y. Chu, Jesse D. Bloom, bioRxiv 2020.12.31.425021; doi: https://doi.org/10.1101/2020.12.31.425021
(8) Nicholas G. Davies, Rosanna C. Barnard, Christopher I. Jarvis, Adam J. Kucharski, James Munday, Carl A. B. Pearson, Timothy W. Russell, Damien C. Tully, Sam Abbott, Amy Gimma, William Waites, Kerry L M Wong, Kevin van Zandvoort, CMMID COVID-19 Working Group, Rosalind M. Eggo, Sebastian Funk, Mark Jit, Katherine E. Atkins, W. John Edmunds. Estimated transmissibility and severity of novel SARS-CoV-2 Variant of Concern 202012/01 in England. medRxiv 2020.12.24.20248822; doi: https://doi.org/10.1101/2020.12.24.20248822
(9) Houriiyah Tegally, Eduan Wilkinson, Marta Giovanetti, et al. Emergence and rapid spread of a new severe acute respiratory syndrome-related coronavirus 2 (SARS-CoV-2) lineage with multiple spike mutations in South Africa. medRxiv 2020.12.21.20248640; doi: https://doi.org/10.1101/2020.12.21.20248640
(10) Kim J S, Jang J H, Kim J M, Chung Y S, Yoo C K, Han M G. Genome-Wide Identification and Characterization of Point Mutations in the SARS-CoV-2 Genome. Osong Public Health Res Perspect. 2020; 11(3):101-111. doi:10.24171/j.phrp.2020.11.3.05
(11) Nilgiriwala K, Mandal A, Patel G, Mestry T, Vaswani S, Shaikh A, Sriraman K, Parikh S, Udupa S, Chatterjee N, Shastri J, Mistry N. Genome Sequences of Five SARS-CoV-2 Variants from Mumbai, India, Obtained by Nanopore Sequencing. Microbiol Resour Announc. 2021 Apr. 15; 10(15):e00231-21

For instance, the mutation N501Y is a non-synonymous mutation within the S-protein's receptor binding domain (RBD) shared by the three SARS-CoV-2 lineages B.1.1.7, P.1 (a.k.a. 20J/501Y.V3) and 501Y.V2 first identified in south eastern England, Brasil/Japan and South Africa respectively. It is one of the key contact residues within the RBD and has been identified as increasing binding affinity to human and murine ACE2. The E484K and E484Q mutation within the S-protein's receptor binding domain (RBD), present in the novel lineages 501Y.S2 and B.1.1.28 from South Africa and Brazil and B1.617.1 from India (Kappa variant of interest) respectively, affect a residue within the RBD that has been shown to be important for binding of many neutralizing antibodies. Accordingly, this mutation affects antibody recognition and enable SARS-CoV-2 immune escape. Virus bearing this mutation has been shown to escape recognition by antibodies in peoples' convalescent sera and may thus alter the effectiveness of vaccines (see e.g. Allison J. Greaney, Andrea N. Loes, Katharine H. D. Crawford, Tyler N. Starr, Keara D. Malone, Helen Y. Chu, Jesse D. Bloom, bioRxiv 2020.12.31.425021). Several other mutations have been discovered, including the K417N, L452R, T478K, and E484K/E484Q mutations. According to the present invention the main naturally occurring mutations thus include, the K417N mutation wherein the amino acid residue (K) at position 417 in SEQ ID NO:2 is substituted by the amino acid residue (N), the L452R mutation wherein the amino acid residue (L) at position 452 in SEQ ID NO:2 is substituted by the amino acid residue (R), the T478K mutation wherein the amino acid residue (T) at position 478 in SEQ ID NO:2 is substituted by the amino acid residue (K) and the E484K or E484Q mutation wherein the amino acid residue (E) at position 484 in SEQ ID NO:2 is substituted by the amino acid residue (K) or (Q), and the N501Y mutation wherein the amino acid residue (N) at position 501 in SEQ ID NO:2 is substituted by the amino acid residue (Y).

As used herein, the term “non-naturally occurring mutation” refers to any mutation that are genetically inserted in the polypeptides of the present invention. In particular, said mutations are inserted to ease the production of the polypeptide. For instance, said mutations include the mutation C538S in SEQ ID NO:2 wherein the amino acid residue (C) at position 538 in SEQ ID NO:2 is substituted by the amino acid residue (S). Said mutations are particularly suitable for avoiding the creation of disulphide bonds within the polypeptide of the present invention.

As used herein, the terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence.

As used herein, the term “promoter/regulatory sequence” refers to a nucleic acid sequence (such as, for example, a DNA sequence) recognized by the synthetic machinery of the cell, or introduced synthetic machinery, required to initiate the specific transcription of a polynucleotide sequence, thereby allowing the expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

As used herein, the term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

As used herein, the term “expression system” means a host cell and compatible vector under suitable conditions, e.g., for the expression of a protein coded for by foreign DNA carried by the vector and introduced to the host cell.

As used herein, the term “conjugate” or interchangeably “conjugated polypeptide” is intended to indicate a composite or chimeric molecule formed by the covalent attachment of one or more polypeptides. The term “covalent attachment” “or “conjugation” means that the polypeptide and the non-peptide moiety are either directly covalently joined to one another, or else are indirectly covalently joined to one another through an intervening moiety or moieties, such as a bridge, spacer, or linkage moiety or moieties. A particular conjugate is a fusion protein.

As used herein, the term “fusion protein” indicates a protein created through the attaching of two or more polypeptides which originated from separate proteins. In particular fusion proteins can be created by recombinant DNA technology and are typically used in biological research or therapeutics. Fusion proteins can also be created through chemical covalent conjugation with or without a linker between the polypeptides portion of the fusion proteins. In the fusion protein the two or more polypeptide are fused directly or via a linker.

As used herein, the term “directly” means that the first amino acid at the N-terminal end of a first polypeptide is fused to the last amino acid at the C-terminal end of a second polypeptide. This direct fusion can occur naturally as described in (Vigneron et al., Science 2004, PMID 15001714), (Warren et al., Science 2006, PMID 16960008), (Berkers et al., J. Immunol. 2015a, PMID 26401000), (Berkers et al., J. Immunol. 2015b, PMID 26401003), (Delong et al., Science 2016, PMID 26912858) (Liepe et al., Science 2016, PMID 27846572), (Babon et al., Nat. Med. 2016, PMID 27798614).

As used herein, the term “linker” has its general meaning in the art and refers to an amino acid sequence of a length sufficient to ensure that the proteins form proper secondary and tertiary structures. In some embodiments, the linker is a peptidic linker which comprises at least one, but less than 30 amino acids, e.g., a peptidic linker of 2-30 amino acids, preferably of 10-30 amino acids, more preferably of 15-30 amino acids, still more preferably of 19-27 amino acids, most preferably of 20-26 amino acids. In some embodiments, the linker has 2; 3; 4; 5; 6; 7; 8; 9; 10; 11; 12; 13; 14; 15; 16; 17; 18; 19; 20; 21; 22; 23; 24; 25; 26; 27; 28; 29; 30 amino acid residues. Typically, linkers are those which allow the compound to adopt a proper conformation (i.e., a conformation allowing a proper signal transducing activity through the IL-15Rbeta/gamma signalling pathway). The most suitable linker sequences (1) will adopt a flexible extended conformation, (2) will not exhibit a propensity for developing ordered secondary structure which could interact with the functional domains of fusion proteins, and (3) will have minimal hydrophobic or charged character which could promote interaction with the functional protein domains.

As used herein, the term “antibody” refers to immunoglobulin molecules and immunologically active portions of immunoglobulin molecules, i.e., molecules that contain an antigen binding site that immunospecifically binds to an antigen. In natural antibodies of rodents and primates, two heavy chains are linked to each other by disulfide bonds, and each heavy chain is linked to a light chain by a disulfide bond. There are two types of light chains, lambda (l) and kappa (k). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non hypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the agonist antibodies described hereafter, the CDRs have been determined using CDR finding algorithms from www.bioinf.org.uk—see the section entitled «How to identify the CDRs by looking at a sequence» within the Antibodies pages.

As used herein, the term “immunoglobulin domain” refers to a globular region of an antibody chain (such as e.g., a chain of a heavy chain antibody or a light chain), or to a polypeptide that essentially consists of such a globular region.

As used herein, the term “Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody. Accordingly, a composition of antibodies of the invention may comprise antibody populations with all K447 residues removed, antibody populations with no K447 residues removed, and antibody populations having a mixture of antibodies with and without the K447 residue.

As used herein, the term “chimeric antibody” refers to an antibody which comprises a VH domain and a VL domain of a non-human antibody, and a CH domain and a CL domain of a human antibody. In one embodiment, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, of an agonist molecule, e.g., CD40 Ligand, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used herein, the term “humanized antibody” include antibodies which have the 6 CDRs of a murine antibody, but humanized framework and constant regions. More specifically, the term “humanized antibody”, as used herein, may include antibodies in which CDR sequences derived from the germline of another mammalian species, such as a mouse, have been grafted onto human framework sequences.

As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, in one embodiment, the term “human monoclonal antibody”, as used herein, is not intended to include antibodies in which CDR sequences derived from the germline of another mammalian species, such as a mouse, have been grafted onto human framework sequences.

As used herein, the term “immune response” refers to a reaction of the immune system to an antigen in the body of a host, which includes generation of an antigen-specific antibody and/or cellular cytotoxic response. The immune response to an initial antigenic exposure (primary immune response) is typically, detectable after a lag period of from several days to two weeks; the immune response to subsequent stimulus (secondary immune response) by the same antigen is more rapid than in the case of the primary immune response. An immune response to a transgene product may include both humoral (e.g., antibody response) and cellular (e.g., cytolytic T cell response) immune responses that may be elicited to an immunogenic product encoded by the transgene. The level of the immune response can be measured by methods known in the art (e.g., by measuring antibody titre).

As used herein the term “APCs” or “Antigen Presenting Cells” denotes cells that are capable of activating T-cells, and include, but are not limited to, certain macrophages, B cells and dendritic cells

As used herein, the term “Dendritic cells” or “DCs” refer to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells).

As used herein, the term “CD40” has its general meaning in the art and refers to human CD40 polypeptide receptor. In some embodiments, CD40 is the isoform of the human canonical sequence as reported by UniProtKB-P25942 (also referred as human TNR5).

As used herein, the term “CD40 agonist antibody” is intended to refer to an antibody that increases CD40 mediated signaling activity in the absence of CD40L in a cell-based assay, such as the B cell proliferation assay. In particular, the CD40 agonist antibody (i) it induces the proliferation of B cell, as measured in vitro by flow cytometric analysis, or by analysis of replicative dilution of CFSE-labeled cells; and/or (ii) induces the secretion of cytokines, such as IL-6, IL-12, or IL-15, as measured in vitro with a dendritic cell activation assay.

As used herein, the term “Langerin” has its general meaning in the art and refers to human C-type lectin domain family 4 member K polypeptide. In some embodiments, Langerin is the isoform of the human canonical sequence as reported by UniProtKB-Q9UJ71 (also referred as human CD207).

As used herein, the term “subject” or “subject in need thereof”, is intended for a human or non-human mammal. Typically the patient is affected or likely to be infected with a Sarbecovirus.

As used herein, the term “Covid-19” refers to the respiratory disease induced by the Severe Acute Respiratory Syndrome coronavirus 2 and its variants.

As used herein, the term “asymptomatic” refers to a subject who experiences no detectable symptoms for the coronavirus infection. As used herein, the term “symptomatic” refers to a subject who experiences detectable symptoms of coronavirus infection. Symptoms of coronavirus infection include: fatigue, anosmia, headache, cough, fever, difficulty to breathe.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

As used herein, the term “vaccination” or “vaccinating” means, but is not limited to, a process to elicit an immune response in a subject against a particular antigen.

As used herein, the term “vaccine composition” is intended to mean a composition which can be administered to humans or to animals in order to induce an immune system response; this immune system response can result in the activation of certain cells, in particular APCs, T lymphocytes and B lymphocytes.

As used herein the term “antigen” refers to a molecule capable of being specifically bound by an antibody or by a T cell receptor (TCR) if processed and presented by MHC molecules. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes).

As used herein, the term “adjuvant” refers to a compound that can induce and/or enhance the immune response against an antigen when administered to a subject or an animal. It is also intended to mean a substance that acts generally to accelerate, prolong, or enhance the quality of specific immune responses to a specific antigen. In the context of the present invention, the term “adjuvant” means a compound, which enhances both innate immune response by affecting the transient reaction of the innate immune response and the more long-lived effects of the adaptive immune response by activation and maturation of the antigen-presenting cells (APCs) especially Dentritic cells (DCs).

As used herein, the expression “therapeutically effective amount” is meant a sufficient amount of the active ingredient of the present invention to induce an immune response at a reasonable benefit/risk ratio applicable to the medical treatment.

As used herein, the term “immune checkpoint inhibitor” has its general meaning in the art and refers to any compound inhibiting the function of an immune inhibitory checkpoint protein. As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA.

### Antibodies of the Present Invention:

The first object of the present invention relates to an antibody that is directed against a surface antigen of an antigen presenting cell wherein:
the heavy chain is conjugated or fused to the Npep2 polypeptide, and
the light chain is conjugated or fused to:
   the RBD polypeptide that comprises that comprises the K417N, L452R, T478K, E484K, and N501Y naturally occurring mutations and that comprises the C538S non naturally occurring mutation or,
   the RBD polypeptide that comprises that comprises the K417N, L452R, T478K, E484Q, and N501Y naturally occurring mutations and that comprises the C538S non naturally occurring mutation

In some embodiments, the heavy chain is conjugated or fused to the Npep2 polypeptide that consists of the amino acid sequence that ranges from the residue at position 276 to the residue at position 411 in SEQ ID NO:1.

In some embodiments, the antibody comprises:
a heavy chain conjugated or fused to the Npep2 polypeptide that consists of the amino acid sequence that ranges from the residue at position 276 to the residue at position 411 in SEQ ID NO:1
and a light chain conjugated or fused to the RBD polypeptide that consists of the amino acid that ranges from the amino acid residue at position 319 to the amino acid residue at position 541 in SEQ ID NO:2 comprising the K417N, L452R, T478K, E484K, and N501Y naturally occurring mutations and the C538S non naturally occurring mutation.

In some embodiments, the antibody comprises:
a heavy chain conjugated or fused to the Npep2 polypeptide that consists of the amino acid sequence that ranges from the residue at position 276 to the residue at position 411 in SEQ ID NO:1
and a light chain conjugated or fused to the RBD polypeptide that consists of the amino acid that ranges from the amino acid residue at position 319 to the amino acid residue at position 541 in SEQ ID NO:2 comprising the K417N, L452R, T478K, E484Q, and N501Y naturally occurring mutations and the C538S non naturally occurring mutation.

In some embodiments, the antibody is an IgG antibody, preferably of an IgG1 or IgG4 antibody, or even more preferably of an IgG4 antibody.

In some embodiments, the antibody is a chimeric antibody, in particular a chimeric mouse/human antibody.

In some embodiments, the antibody is humanized antibody.

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In some embodiments, the antibody is a human antibody.

Human antibodies can be identified using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.” The HuMAb mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., 1994 Nature 368(6474): 856-859). In another embodiment, human anti-PD-1 antibodies can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

In some embodiments, the antibody is specific for a cell surface marker of a professional APC. The antibody may be specific for a cell surface marker of another professional APC, such as a B cell or a macrophage.

In some embodiments, the antibody is selected from an antibody that specifically binds to DC immunoreceptor (DCIR), MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-7 receptor and IL-2 receptor, ICAM-1, Fey receptor, LOX-1, and ASPGR.

In some embodiments, the antibody is specific for CD40.

In some embodiments, the anti-CD40 antibody derives from the 12E12 antibody and comprises:
a heavy chain comprising the complementarity determining regions CDR1H, CDR2H and CDR3H, the CDR1H having the amino acid sequence GFTFSDYYMY (SEQ ID NO:3), the CDR2H having the amino acid sequence YINSGGGSTYYPDTVKG (SEQ ID NO:4), and the CDR3H having the amino acid sequence RGLPFHAMDY (SEQ ID NO:5),
and a light chain comprising the complementarity determining regions CDR1L, CDR2L and CDR3L, the CDR1L having the amino acid sequence SASQGISNYLN (SEQ ID NO:6) the CDR2L having the amino acid sequence YTSILHS (SEQ ID NO:7) and the CDR3L having the amino acid sequence QQFNKLPPT (SEQ ID NO: 8).

In some embodiments, the anti-CD40 antibody derives from the 11B6 antibody and comprises:
a heavy chain comprising the complementarity determining regions CDR1H, CDR2H and CDR3H, the CDR1H having the amino acid sequence GYSFTGYYMH (SEQ ID NO:9), the CDR2H having the amino acid sequence RINPYNGATSYNQNFKD (SEQ ID NO:10), and the CDR3H having the amino acid sequence EDYVY (SEQ ID NO:11), and
a light chain comprising the complementarity determining regions CDR1L, CDR2L and CDR3L, the CDR1L having the amino acid sequence RSSQSLVHSNGNTYLH (SEQ ID NO:12) the CDR2L having the amino acid sequence KVSNRFS (SEQ ID NO:13) and the CDR3L having the amino acid sequence SQSTHVPWT (SEQ ID NO:14).

In some embodiments, the anti-CD40 antibody derives from the 12B4 antibody and comprises:
a heavy chain comprising the complementarity determining regions CDR1H, CDR2H and CDR3H, the CDR1H having the amino acid sequence GYTFTDYVLH (SEQ ID NO:15), the CDR2H having the amino acid sequence YINPYNDGTKYNEKFKG (SEQ ID NO:16), and the CDR3H having the amino acid sequence GYPAYSGYAMDY (SEQ ID NO:17), and
a light chain comprising the complementarity determining regions CDR1L, CDR2L and CDR3L, the CDR1L having the amino acid sequence RASQDISNYLN (SEQ ID NO:18) the CDR2L having the amino acid sequence YTSRLHS (SEQ ID NO:19) and the CDR3L having the amino acid sequence HHGNTLPWT (SEQ ID NO:20).

In some embodiments, the anti-CD40 antibody is selected from the group consisting of selected mAb1, mAb2, mAb3, mAb4, mAb5 and mAb6 as described in Table A.

TABLE A
CD40 antibodies

| | | |
|---|---|---|
| mAb1 [11B6 VH/VkV2] | SEQ ID NO: 21 | SEQ ID NO: 22 |
| mAb2 [11B6 VHV3/VkV2] | SEQ ID NO: 23 | SEQ ID NO: 22 |
| mAb3 [12B4] | SEQ ID NO: 24 | SEQ ID NO: 25 |
| mAb4 [24A3] | SEQ ID NO: 26 | SEQ ID NO: 27 |
| mAb5 [CP870,893] | SEQ ID NO: 28 | SEQ ID NO: 29 |
| mAb6 [12E12] | SEQ ID NO: 30 | SEQ ID NO: 31 |

(Amino acid sequence of variable heavy chain
region (VH) (v2) of Humanized 11B6)
SEQ ID NO: 21
EVQLVQSGAEVKKPGASVKISCKASGYSFTGYYMHWVKQAHGQGLEWIG
RINPYNGATSYNQNFKDRATLTVDKSTSTAYMELSSLRSEDTAVYYCAR
EDYVYWGQGTTVTVSSAS
(Amino acid sequence of variable light chain (VL)
Vk (v2) of humanized 11B6 VL)
SEQ ID NO: 22
DVVMTQSPLSLPVTLGQPASISCRSSQSLVHSNGNTYLHWYQQRPGQSP
RLLIYKVSNRFSGVPDRFSGSGSGTDFTLKISRVEAEDVGVYFCSQSTH
VPWTFGGGTK
(Amino acid sequence of variable heavy chain
region VH (v3) of humanized 11B6)
SEQ ID NO: 23
EVQLVQSGAEVKKPGASVKVSCKASGYSFTGYYMHWVRQAPGQGLEWIG
RINPYNGATSYNQNFKDRVTLTVDKSTSTAYMELSSLRSEDTAVYYCAR
EDYVYWGQGTTVTVSSAS
(VH amino acid sequence of mAb3 (12B4))
SEQ ID NO: 24
EVQLQQSGPELVKPGASVKMSCKASGYTFTDYVLHWVKQKPGQGLEWIG
YINPYNDGTKYNEKFKGKATLTSDKSSSTAYMELSSLTSEDSAVYYCAR
GYPAYSGYAMDYWGQGTSVTVSSAS
(VL amino acid sequence of mAb3 (12B4))
SEQ ID NO: 25
DIQMTQTTSSLSASLGDRVTISCRASQDISNYLNWYQQKPDGTVKLLIY
YTSRLHSGVPSRFSGSGSGTDYSLTISNLEQEDIATYFCHHGNTLPWTF
GGGTK
VH amino acid sequence of mAb4 (24A3 HC))
SEQ ID NO: 26
DVQLQESGPDLVKPSQSLSLTCTVTGYSITSDYSWHWIRQFPGNKLEWM
GYIYYSGSTNYNPSLKSRISITRDTSKNQFFLQLNSVTTEDSATYFCAR
FYYGYSFFDYWGQGTTLTVSSAS
(VL amino acid sequence of mAb4 (24A3 KC))
SEQ ID NO: 27
QIVLTQSPAFMSASPGEKVTMTCSASSSVSYMHWYQQKSGTSPKRWIYD
TSKLASGVPARFSGSGSGTSYSLTISSMEAEDAATYYCQQWSSNPLTFG
AGTK
(VH amino acid sequence of mAb5)
SEQ ID NO: 28
QVQLVQSGAEVKKPGASVKVSCKASGYTFTGYYMHWVRQAPGQGLEWMG
WINPDSGGTNYAQKFQGRVTMTRDTSISTAYMELNRLRSDDTAVYYCAR
DQPLGYCTNGVCSYFDYWGQGTLVTVSSAS
(VL amino acid sequence of mAb5)
SEQ ID NO: 29
DIQMTQSPSSVSASVGDRVTITCRASQGIYSWLAWYQQKPGKAPNLLIY
TASTLQSGVPSRFSGSGSGTDFTLTISSLQPEDFATYYCQQANIFPLTF
GGGTK
(VH amino acid sequence of mAb6 (12E12 H3
Humanized HC))
SEQ ID NO: 30
EVQLVESGGGLVQPGGSLKLSCATSGFTFSDYYMYWVRQAPGKGLEWVA
YINSGGGSTYYPDTVKGRFTISRDNAKNTLYLQMNSLRAEDTAVYYCAR
RGLPFHAMDYWGQGTLVTVSSAS
(VL amino acid sequence of mAb6 (Humanized
K2 12E12))
SEQ ID NO: 31
DIQMTQSPSSLSASVGDRVTITCSASQGISNYLNWYQQKPGKAVKLLIY
YTSILHSGVPSRFSGSGSGTDYTLTISSLQPEDFATYYCQQFNKLPPTF
GGGTK

In some embodiments, the anti-CD40 antibody is selected from the group consisting of:
an antibody comprising a heavy chain having wherein the variable domain has a sequence set forth as SEQ ID NO: 21 and a light chain wherein the variable domain has a sequence set forth as SEQ ID NO: 22,
an antibody comprising a heavy chain wherein the variable domain has a sequence set forth as SEQ ID NO: 23 and a light chain wherein the variable domain has a sequence set forth as SEQ ID NO: 22,
an antibody comprising a heavy chain wherein the variable domain has a sequence set forth as SEQ ID NO: 24 and a light chain wherein the variable domain has a sequence set forth as SEQ ID NO: 25,
an antibody comprising a heavy chain wherein the variable domain has a sequence set forth as SEQ ID NO: 26 and a light chain wherein the variable domain has a sequence set forth as SEQ ID NO: 27,
an antibody comprising a heavy chain wherein the variable domain has a sequence set forth as SEQ ID NO: 28 and a light chain wherein the variable domain has a sequence set forth as SEQ ID NO: 29, and
an antibody comprising a heavy chain wherein the variable domain has a sequence set forth as SEQ ID NO: 30 and a light chain wherein the variable domain has a sequence set forth as SEQ ID NO: 31.

In some embodiments, the anti-CD40 antibody is a CD40 agonist antibody. CD40 agonist antibodies are described in WO2010/009346, WO2010/104747 and WO2010/104749. Other anti-CD40 agonist antibodies in development include CP-870,893 that is a fully human IgG2 CD40 agonist antibody developed by Pfizer. It binds CD40 with a KD of 3.48×10−10 M, but does not block binding of CD40L (see e.g., U.S. Pat. No. 7,338,660) and SGN-40 that is a humanized IgG1 antibody developed by Seattle Genetics from mouse antibody clone S2C6, which was generated using a human bladder carcinoma cell line as the immunogen. It binds to CD40 with a KD of 1.0×10-9 M and works through enhancing the interaction between CD40 and CD40L, thus exhibiting a partial agonist effect (Francisco J A, et al., Cancer Res, 60: 3225-31, 2000). Even more particularly, the CD40 agonist antibody is selected from the group consisting of selected mAb1, mAb2, mAb3, mAb4, mAb5 and mAb6 as described in Table A.

In some embodiments, the antibody is specific for Langerin. In some embodiments, the antibody derives from the antibody 15B10 having ATCC Accession No. PTA-9852. In some embodiments, the antibody derives from the antibody 2G3 having ATCC Accession No. PTA-9853. In some embodiments, the antibody derives from the antibody 91E7, 37C1, or 4C7 as described in WO2011032161.

In some embodiments, the anti-Langerin antibody comprises a heavy chain comprising the complementarity determining regions CDR1H, CDR2H and CDR3H of the 15B10 antibody and a light chain comprising the complementarity determining regions CDR1L, CDR2L and CDR3L of the 15B10 antibody.

In some embodiments, the anti-Langerin antibody comprises a heavy chain comprising the complementarity determining regions CDR1H, CDR2H and CDR3H of the 2G3 antibody and a light chain comprising the complementarity determining regions CDR1L, CDR2L and CDR3L of the 2G3 antibody.

In some embodiments, the anti-Langerin antibody comprises a heavy chain comprising the complementarity determining regions CDR1H, CDR2H and CDR3H of the 4C7 antibody and a light chain comprising the complementarity determining regions CDR1L, CDR2L and CDR3L of the 4C7 antibody.

In some embodiments, the anti-Langerin antibody is selected from the group consisting of selected mAb7, mAb8, mAb9, as described in Table B.

| | | |
|---|---|---|
| mAb7 [15B10] | SEQ ID NO: 32 | SEQ ID NO: 33 |
| mAb8 [2G3] | SEQ ID NO: 34 | SEQ ID NO: 35 |
| mAb9 [4C7] | SEQ ID NO: 36 | SEQ ID NO: 37 |

(Amino acid sequence of variable heavy chain
region (VH) of 15B10)
SEQ ID NO: 32
SVKMSCKASGYTFTDYVISWVKQRTGQGLEWIGDIYPGSGYSFYNENFK
GKATLTADKSSTTAYMQLSSLTSEDSAVYFCA
(Amino acid sequence of variable light chain (VL)
15B10)
SEQ ID NO: 33
ASISCRSSQSLVHSNGNTYLHWYLQKPGQSPKLLIYKVSNRFSGVPDRF
SGSGSGTNFTLKISRVEAEDLGLYFCS
(Amino acid sequence of variable heavy chain
region (VH) of 2G3)
SEQ ID NO: 34
SSVKMSCKASGYTFTDYVISWVKQRTGQGLEWIGDIYPGSGYSFYNENF
KGKATLTADKSSTTAYMQLSSLTSEDSAVYFCA
(Amino acid sequence of variable light chain (VL)
2G3)
SEQ ID NO: 35
VTLTCRSSTGAVTTSNYANWVQEKPDHLFTGLIGGTNNRVSGVPARFSG
SLIGDKAALTITGAQTEDEAIYFCA
(Amino acid sequence of the heavy chain of 4C7)
SEQ ID NO: 36
QVQLQQSGAELVRPGASVTLSCKASGYTFIDHDMHWVQQTPVYGLEWIG
AIDPETGDTGYNQKFKGKAILTADKSSRTAYMELRSLTSEDSAVYYCTI
PFYYSNYSPFAYWGQGALVTVSAAKTTAPSVYPLAPVCGGTTGSSVTLG
CLVKGYFPEPVTLTWNSGSLSSGVHTFPALLQSGLYTLSSSVTVTSNTW
PSQTITCNVAHPASSTKVDKKIEPRVPITQNPCPPLKECPPCADLLGGP
SVFIFPPKIKDVLMISLSPMVTCVVVDVSEDDPDAQISWFVNNVEVHTA
QTQTHREDYNSTLRVVSALPIQHQDWMSGKEFKCKVNNRALPSPIEKTI
SKPRGPVRAPQVYVLPPPAEEMTKKEFSLTCMITGFLPAEIAVDWTSNG
RTEQNYKNTATVLDSDGSYFMYSKLRVQKSTWERGSLFACSVVHEGLHN
HLTTKTISRSLGKAS
(Amino acid sequence of light chain of 4C7)
SEQ ID NO: 37
QIVLSQSPAILSASPGEKVTMTCRASSSVSYMHWYQRKPGSSPKPWIYA
TSNLASGVPARFSGSGSGTSYSLTISRVEAEDAATYYCQQWSSNPLTFG
AGTKLELKRADAAPTVSIFPPSSEQLTSGGASVVCFLNNFYPKDINVKW
KIDGSERQNGVLNSWTDQDSKDSTYSMSSTLTLTKDEYERHNSYTCEAT
HKTSTSPIVKSFNRNEC

In some embodiments, the anti-Langerin antibody is selected from the group consisting of:
an antibody comprising a heavy chain having wherein the variable domain has a sequence set forth as SEQ ID NO: 32 and a light chain wherein the variable domain has a sequence set forth as SEQ ID NO: 33,
an antibody comprising a heavy chain wherein the variable domain has a sequence set forth as SEQ ID NO: 34 and a light chain wherein the variable domain has a sequence set forth as SEQ ID NO: 35, and
an antibody comprising a heavy chain having a sequence set forth as SEQ ID NO: 36 and a light chain having a sequence set forth as SEQ ID NO: 37.

The antibodies of the invention may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides. For instance, the antibodies of the invention can be synthesized by recombinant DNA techniques as is now well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

In some embodiments, the light chain is conjugated or fused to the RBD polypeptide via its C-terminus. In some embodiments, the light chain of the antibody is fused to the N-terminus of the RBD polypeptide.

In some embodiments, the heavy chain is conjugated or fused to the Npep2 polypeptide via its C-terminus. In some embodiments, the heavy chain of the antibody is fused to the N-terminus of the Npep2 polypeptide.

In some embodiments, the heavy chain and/or the light chain of the antibody is conjugated to the RBD polypeptide by using chemical coupling. Several methods are known in the art for the attachment or conjugation of an antibody to its conjugate moiety. Examples of linker types that have been used to conjugate a moiety to an antibody include, but are not limited to, hydrazones, thioethers, esters, disulfides and peptide-containing linkers, such as valine-citruline linker. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). Techniques for conjugating polypeptides and in particular, are well-known in the art (See, e.g., Amon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58; see also, e.g., PCT publication WO 89/12624.) Typically, the peptide is covalently attached to lysine or cysteine residues on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target human epidermal growth factor receptor 2-positive breast cancer. Clin. Cancer Res.16, 4769-4778). Junutula et al. (Nat Biotechnol. 2008; 26:925-32) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

In some embodiments, the conjugation is carried out by a dockerin domain or multiple domains to permit non-covalent coupling to cohesin fusion proteins as described in US20160031988A1 and US20120039916A1.

In some embodiments, the fusion is carried out either directly or via a linker. As used herein, the term “directly” means that the first amino acid at the N-terminal end of a first polypeptide is fused to the last amino acid at the C-terminal end of a second polypeptide. This direct fusion can occur naturally as described in (Vigneron et al., Science 2004, PMID 15001714), (Warren et al., Science 2006, PMID 16960008), (Berkers et al., J. Immunol. 2015a, PMID 26401000), (Berkers et al., J. Immunol. 2015b, PMID 26401003), (Delong et al., Science 2016, PMID 26912858) (Liepe et al., Science 2016, PMID 27846572), (Babon et al., Nat. Med. 2016, PMID 27798614).

In some embodiments, the linker is selected from the group consisting of FlexV1, f1, f2, f3, or f4 as described below.

In other words, in some embodiments, the linker is selected from the group consisting of SEQ ID NO:38, SEQ ID NO:39, SEQ ID NO:40, SEQ ID NO:41 and SEQ ID NO:42.

(flexV1, SEQ ID NO: 38)
QTPTNTISVTPTNNSTPTNNSNPKPNP
(f1, SEQ ID NO: 39)
SSVSPTTSVHPTPTSVPPTPTKSSP
(f2, SEQ ID NO: 40)
PTSTPADSSTITPTATPTATPTIKG
(f3, SEQ ID NO: 41)
TVTPTATATPSAIVTTITPTATTKP
(f4, SEQ IDNO: 42)
TNGSITVAATAPTVTPTVNATPSAA

In some embodiments, the fusion protein of the present invention further comprises one or more sequences originating from the restriction cloning site(s) present in the polynucleotide encoding for said fusion protein. Typically, said sequences may consist of 2 amino acid residues and typically include AP, AS, AR, PR, SA, TR, and TS sequences.

In some embodiments, the fusion protein comprises the sequence of a signal peptide. As used herein, the term “signal peptide” has its general meaning in the art and refers to a pre-peptide which is present as an N-terminal peptide on a precursor form of a protein. The function of the signal peptide is to facilitate translocation of the expressed polypeptide to which it is attached into the endoplasmic reticulum. The signal peptide is normally cleaved off in the course of this process. The signal peptide may be heterologous or homologous to the organism used to produce the polypeptide.

In some embodiments, the antibody comprises i) a light chain that is fused to the RBD polypeptide to form the fusion protein as set forth in SEQ ID NO:43 and ii) the heavy chain that is fused to the Npep2 polypeptide to form the fusion protein as set forth in SEQ ID NO: 45.

In some embodiments, the antibody comprises i) a light chain that is fused to the RBD polypeptide to form the fusion protein as set forth in SEQ ID NO:44 and the heavy chain that is fused to the Npep2 polypeptide to form the fusion protein as set forth in SEQ ID NO:45.

SEQ ID NO: 43>[hAnti-CD40VK2-LV-hIgGK-C-ViralSARS-COV-2-Spike-RBDC221S SAD-K
var]. Variable domain; LE (joining region between variable and constant
domains); Constant domain; AS areas with restriction sites for cloning;

[symbol]

[symbol]

DIQMTQSPSSLSASVGDRVTITCSASQGISNYLNWYQQKPGKAVKLLIYYTSILHSGVPSRFSGSGSGTDYTLTI
SSLQPEDFATYYCQQFNKLPPTFGGGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGECAS

[symbol]

[symbol]

[symbol]

[symbol]

SEQ ID NO: 44>[hAnti-CD40VK2-LV-hIgGK-C-ViralSARS-COV-2-Spike-RBDC221S SAD-K
var]. Variable domain; LE (joining region between variable and constant
domains); Constant domain; AS areas with restriction sites for cloning;

[symbol]

[symbol]

DIQMTQSPSSLSASVGDRVTITCSASQGISNYLNWYQQKPGKAVKLLIYYTSILHSGVPSRFSGSGSGTDYTLTI
SSLQPEDFATYYCQQFNKLPPTFGGGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGECAS

[symbol]

[symbol]

[symbol]

[symbol]

SEQ ID NO: 45> [hAnti-CD40VH3-LV-hIgG4H-C-f4-ViralSARS-Cov-2-Npep2] Variable
domain; TKGP (joining region between variable and constant domains); Constant
domain; AS restriction site to join the fusion antigen; linker f4;

[symbol]

[symbol]

EVQLVESGGGLVQPGGSLKLSCATSGFTFSDYYMYWVRQAPGKGLEWVAYINSGGGSTYYPDTVKGRFTISRDNA
KNTLYLQMNSLRAEDTAVYYCARRGLPFHAMDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTYTCNVDHKPSNTKVDKRVESKYGPPC
PPCPAPEFEGGPSVELFPPKPKDTLMISRTPEVTCVVVDVSQEDPEVQENWYVDGVEVHNAKTKPREEQENSTYR
VVSVLTVLHQDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVESCSVMHEALHNHYTQKSLSLSLGKASTN
GSITVAATAPTVTPTVNATPSAAAS

[symbol]

[symbol]

[symbol]

### Nucleic Acids, Vectors and Host Cells of the Present Invention:

A further object of the invention relates to a nucleic acid that encodes for a heavy chain and/or the light chain of the antibody of the present invention.

Typically, said nucleic acid is a DNA or RNA molecule, which may be included in any suitable vector, such as a plasmid, cosmid, episome, artificial chromosome, phage or a viral vector.

So, a further object of the invention relates to a vector comprising a nucleic acid of the present invention.

Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a host cell which has been transfected, infected or transformed by a nucleic acid and/or a vector according to the invention.

The nucleic acids of the invention may be used to produce an antibody of the present invention in a suitable expression system. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts. Mammalian host cells include Chinese Hamster Ovary (CHO cells) including dhfr− CHO cells (described in Urlaub and Chasin, 1980) used with a DHFR selectable marker, CHOK1 dhfr+ cell lines, NSO myeloma cells, COS cells and SP2 cells, for example GS CHO cell lines together with GS Xceed™ gene expression system (Lonza), or HEK cells.

The present invention also relates to a method of producing a recombinant host cell expressing a polypeptide according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

The host cell as disclosed herein are thus particularly suitable for producing the antibody of the present invention. Indeed, when recombinant expression are introduced into mammalian host cells, the polypeptides are produced by culturing the host cells for a period of time sufficient for expression of the antibody in the host cells and, optionally, secretion of the antibody into the culture medium in which the host cells are grown. The antibodies can be recovered and purified for example from the culture medium after their secretion using standard protein purification methods.

### Pharmaceutical and Vaccine Compositions:

The antibodies as described herein may be administered as part of one or more pharmaceutical compositions. Except insofar as any conventional carrier medium is incompatible with the antibodies of the present invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The antibodies as described herein are particularly suitable for preparing vaccine composition. Thus a further object of the present invention relates to a vaccine composition comprising an antibody of the present invention.

In some embodiments, the vaccine composition of the present invention comprises an adjuvant. In some embodiments, the adjuvant is alum. In some embodiments, the adjuvant is Incomplete Freund's adjuvant (IFA) or other oil based adjuvant that is present between 30-70%, preferably between 40-60%, more preferably between 45-55% proportion weight by weight (w/w). In some embodiments, the vaccine composition of the present invention comprises at least one Toll-Like Receptor (TLR) agonist which is selected from the group consisting of TLR1, TLR2, TLR3, TLR4, TLR5, TLR6, TLR7, and TLR8 agonists.

### Therapeutic Methods:

The antibodies as well as the pharmaceutical or vaccine compositions as herein described are particularly suitable for inducing an immune response against Sarbecoviruses and especially against SARS-CoV-2 and thus can be used for vaccine purposes. The antibodies of the present invention can be used as universal Sarbecovirus vaccines.

Therefore, a further object of the present invention relates to a method for vaccinating a subject in need thereof against Sarbecoviruses comprising administering a therapeutically effective amount of the antibody of the present invention.

A further object of the present invention relates to a method for vaccinating a subject in need thereof against SARS-CoV-2 comprising administering a therapeutically effective amount of the antibody of the present invention.

In some embodiments, the antibodies as well as the pharmaceutical or vaccine compositions as herein described are particularly suitable for the treatment of Covid-19.

In some embodiments, the subject can be human or any other animal (e.g., birds and mammals) susceptible to coronavirus infection (e.g., domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

In some embodiments, the subject can be symptomatic or asymptomatic.

Typically, the active ingredient of the present invention (i.e., the antibodies and the pharmaceutical or vaccine compositions as herein described) is administered to the subject at a therapeutically effective amount. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; the activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The antibodies and the pharmaceutical or vaccine compositions as herein described may be administered to the subject by any route of administration and in particular by oral, nasal, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active agent which is being used.

In some embodiments, the antibodies as well as the pharmaceutical or vaccine compositions as herein described may be administered to the subject in combination with, for example, any known therapeutic agent or method for vaccinating against SARS-CoV-2 coronavirus. Non-limiting examples of such known therapeutics include but are not limited to anti-viral agents such as remdesivir, lopinavir, ritonavir, hydroxycholoroquine, and chloroquine. In some embodiments, the Antibodies and the pharmaceutical or vaccine compositions as herein described are administered in combination with an immune checkpoint inhibitor. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist. In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

The invention will be further illustrated by the following figures and examples. However, these examples and figures should not be interpreted in any way as limiting the scope of the present invention.

### FIGURES

FIG. 1: Design of Pan-Sarbecovirus DC-targeting vaccines (CD40.Pan.CoV).

FIG. 2: Pan-Sarbecovirus DC-targeting vaccine tested via in vitro expansion of Sarbecovirus T cells in SARS-CoV-2-infected convalescent donor PBMC cultures. A) SARS-CoV-2+ convalescent donor patient PBMCs (n=6) were stimulated at DO with CD40.N2.RBDvSAD vaccine (1 nM), cultured for 9 days with IL-2, and then stimulated in the last 24 h with different overlapping peptide (OLP) pools (1 μg/ml) spanning the following sequences: i) vaccine RBD (vRBD) of SARS-CoV-2 (Wuhan), VOC beta/gamma, VOC Delta, SARS-CoV-1, ii) vaccine Npep2 (vN) of SARS-CoV-2 and SARS-CoV-1 regions, then analysed at D9 by intracellular cytokine staining (ICS). B) The graphs show stacked values for % of CD4+ T and CD8+ T cells optionally stained for intracellular interferon γ (IFNγ+), intracellular Tumor Necrosis Factor α (TNFα+), or Interleukin 2 (IL-2+).

FIG. 3: Analysis of the homologies of the vaccine RDB and Npep2 (N2) regions among Betacoronaviruses revealed a high percentage of homologies of these vaccine regions within the Sarbecoviruses. We used Cobalt (https://www-ncbi-nlm-nih-gov.proxy.insermbiblio.inist.fr/tools/cobalt/re_cobalt.cgi) to perform the alignment of sequences from SARS-CoV-2, four SARS-CoV-2 VOCs (α, β, γ, δ), SARS-CoV-1, and 32 recently described SARS-CoV-related coronaviruses which include 30 viruses of bat origin and two of pangolin origin (all from the Sarbecovirus subgenus).

FIG. 4: Pan-Sarbecovirus DC-targeting vaccine (CD40.Pan.CoV) induced in vitro activation and plasma cell differentiation of B cells from SARS-CoV-2-infected convalescent donor PBMC. A) PBMCs from COVID-19 convalescent individuals were stimulated in vitro with CD40.CoV2, CD40.Pan.CoV (i.e CD40.N2.RBDvSAD-Q), IgG4.CoV2, CD40.NivG (Nipah control vaccine) or non-fused CD40 mAb (1 nM). PBMCs (5E05) were incubated in 300 μL of RPMI supplemented with 10% human serum AB (SAB). After 48 hours, the supernatant is discarded and replaced by 1 mL of fresh RPMI supplemented with 10% human serum AB (SAB). The medium is replenished by half every 2 days. After 7 days, PBMCs are washed in RPMI 10% FCS and cultured in 500 ul of RPMI 10% FCS. B) After 9 days, cells were stained with viability marker in addition to CD19 Alexa700 (from BD; ref:561031), CD21 PECF594 (from BD; ref:563474), CD27 APC (from BD; ref 337169), CD38 PercPCy5.5 (from Biolegend; ref 303522), CD138 PE (from BD; ref: 552026), and IgD FITC (from Invitrogen; ref: H15501) to determine B cells lineages and analyzed using an LSR II-3 laser flow cytometer (405, 488, and 640 nm) (Becton Dickinson). C) Measurement of IgG antibody directed against SARS-CoV-2 RBD in cell culture supernatant were performed using LUMINEX technology.

FIG. 5: Pan-Sarbecovirus DC-targeting vaccine (CD40.Pan.CoV) protected K18 hACE2tg×hCD40tg mice from SARS-CoV2 infection. K18 hACE2tg×hCD40tg mice were immunized twice, at day 0 and day 21, intraperitonally with the CD40.Pan.CoV vaccine (10 μg/mouse)+poly-ICLC (Hiltonol®) as an adjuvant (50 μg/mouse) (n=6). Mock mice received PBS or poly-ICLC at D0 and D21 (n=9/group). Mice were then infected with SARS-CoV2 virus (Wuhan strain 10\hat{ }4 PFU) through intranasal instillation at D28 post prime. A) Mice were monitored for survival for 12 days. B) Three mice per group were randomly selected and sacrificed on day 5 post-infection to evaluate the lungs' viral load and viral infectivity.

FIG. 6: Antiviral efficacy of Pan-Sarbecovirus DC-targeting vaccine (CD40.Pan.CoV) in convalescent NHPs challenged with a Delta SARS-CoV-2 strain. We subcutaneously injected five convalescent cynomolgus macaques with 200 μg of the vaccine without adjuvant. All the convalescent macaques were previously exposed to SARS-CoV-2 (1×10\hat{ }6 PFU; strain: BetaCoV/France/IDF/0372/2020; March-April 2020 and October 2020) and confirmed to be infected by RT-qPCR and serology. Four weeks after immunization, the five CD40.Pan.CoV-immunized convalescent NHPs and six SARS-CoV-2-naïve animals (control group) were exposed to a total dose of 10e5 TCID₅₀ of SARS-CoV-2 B.1.617.2 variant virus (Delta strain; SARS-CoV-2, hCoV-19/USA/PHC658/2021; BEI Resources Repository (National Instituted of Health, USA)) via the combination of intranasal and intratracheal routes 25 (0.25 mL in each nostril and 4.5 mL in the trachea, i.e. a total of 5 mL; day 0), using atropine (0.04 mg/kg) for pre-medication and ketamine (5 mg/kg) with medetomidine (0.05 mg/kg) for anesthesia. Blood, nasopharyngeal & tracheal swabs and bronchoalveolar lavage were regularly collected. A) Genomic viral RNA (gRNA) quantification in tracheal, nasopharyngeal swabs and BAL of naïve and CD40.Pan.CoV-vaccinated convalescent macaques. The horizontal dotted line represents the limit of detection. B) Subgenomic viral RNA (gRNA) quantification in tracheal, nasopharyngeal swabs and BAL of naïve and CD40.Pan.CoV-vaccinated convalescent macaques. The horizontal dotted line represents the limit of detection.

FIG. 7: Immunogenicity of Pan-Sarbecovirus DC-targeting vaccine (CD40.Pan.CoV) in convalescent NHPs. A) Anti-Spike (S), B) anti-Nucleocapside (N) and C) anti-RBD Delta, D) anti-RBD Wuhan or E) anti-RBD Beta IgG were titrated using a commercially available multiplexed immunoassay developed by Mesoscale Discovery (MSD, Rockville, MD) as previously described (Anderson, E. J. et al. Safety and immunogenicity of SARS-CoV-2 mRNA-1273 vaccine in older adults. N. Engl. J. Med. 383, 2427-2438 (2020)). A) Anti-Spike and C) anti-RBD Delta, D) anti-RBD Wuhan and E) anti-RBD Beta antibodies neutralizing the binding of the spike protein to the ACE2 receptor were titrated with the MSD pseudo-neutralization assay.

### EXAMPLE

Methods for expression vectors and protein purification production and quality assurance including CD40 binding specificity were as are described [1; 2; 3, 4, 5]. Protein expression was via transient CHO-S(Chinese Hamster Ovary cells) transfection using the TransIT®-CHO Transfection Kit (Mirus). Clonings used synthetic DNA cassettes encoding, using CHO-optimized codons), the various SARS-CoV-2 antigen regions, typically bounded by restriction sites convenient for ligations to vectors in various combinations.
[1] Flamar A L., S. Zurawski, F. Scholz, I. Gayet, L. Ni, XH. Li, E. Klechevsky, J. Quinn, S. Oh, D. H. Kaplan, J. Banchereau and G. Zurawski. 2012. Noncovalent assembly of anti-Dendritic Cell antibodies and antigens for evoking immune responses in vitro and in vivo. J. Immunl. 189: 2645-55.
[2] Flamar A L., Y. Xue, S. M. Zurawski, M. Montes, B. King, L. Sloan, S. Oh, J. Banchereau, Y. Levy and G. Zurawski. 2013. Targeting concatenated HIV antigens to human CD40 expands a broad repertoire of multifunctional CD4+ and CD8+ T Cells. AIDS. 27: 2041-51.
[3] Zurawski G., X. Shen, S. Zurawaski, G. D. Tomaras, D. C. Montafiori, M Roederer, G. Ferrari, C. Lacabaratz, P. Klucar, Z. Wang, K. E. Foulds, SF. Kao, X. Yu, A. Sato, N. L. Yates, C. LaBranche, S. Stanfield-Oakley, K. Kibler, B. Jacobs, A. Salazar, S. Self, E. Fulp, R. Gottardo, L. Galmin, D. Weiss, A. Cristillo, G. Pantaleo and Y. Levy. 2017. Superiority in rhesus macaques of targeting HIV-1 Env gp140 to CD40 versus LOX-1 in combination with replication-competent NYVAC-KC for induction of Env-specific antibody and T Cell responses. J. Virol. 91: 1-20.
[4] Marlin R., Godot V, Cardinaud S., Galhaut M., Coleon S., Zurawski S., Dereuddre-Bosquet N., Cavarelli M., Gallouet A. S., Maisonnasse P., Dupaty L., Fenwick C., Naninck T., Lemaitre J., Gomez-Pacheco M, Kahlaoui N., Contreras V., Relouzat F., Fang R. H. T., Wang Z., Ellis J. 3rd, Chapon C., Centlivre M., Wiedemann A., Lacabaratz C., Surenaud M., Szurgot I., Liljestrom P., Planas D., Bruel T., Schwartz O., Werf S. V, Pantaleo G., Prague M., Thiebaut R., Zurawski G., Levy Y., Le Grand R. Targeting SARS-CoV-2 receptor-binding domain to cells expressing CD40 improves protection to infection in convalescent macaques. Nature communications. 2021; 12(1):5215.
[5] Coléon S., Wiedemann A., Surénaud M, Lacabaratz C., Hue S., Prague M., Cervantes-Gonzalez M., Wang Z., Ellis J., Sansoni A., Pierini C., Bardin Q., Fabregue M., Sharkaoui S., Hoest P., Dupaty L., Picard F., Centlivre M, Ghosn J., French COVID Cohort Study Group, Thiébaut R., Cardinaud S., Malissen B., Zurawski G., Zarubica A., Zurawski S. M., Godot V., Lévy Y. Design, immunogenicity, and efficacy of a pan-sarbecovirus dendritic-cell targeting vaccine. EBioMedicine. 2022; 80:104062.

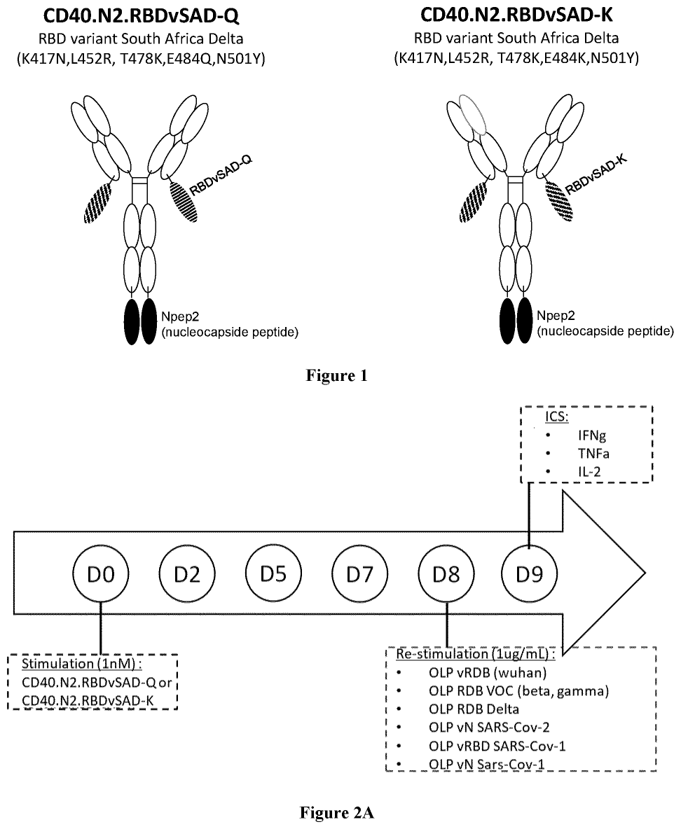

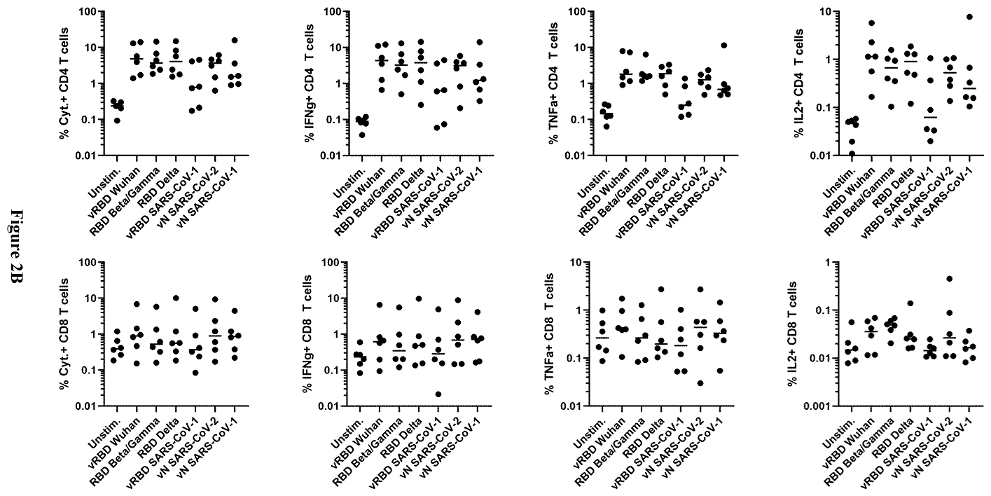

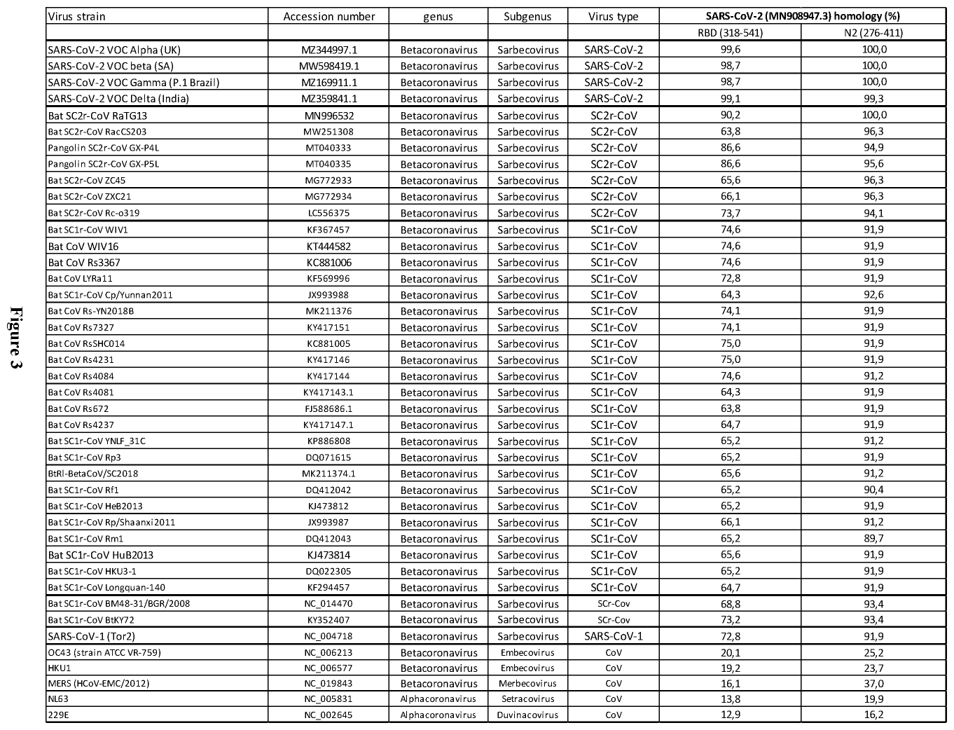

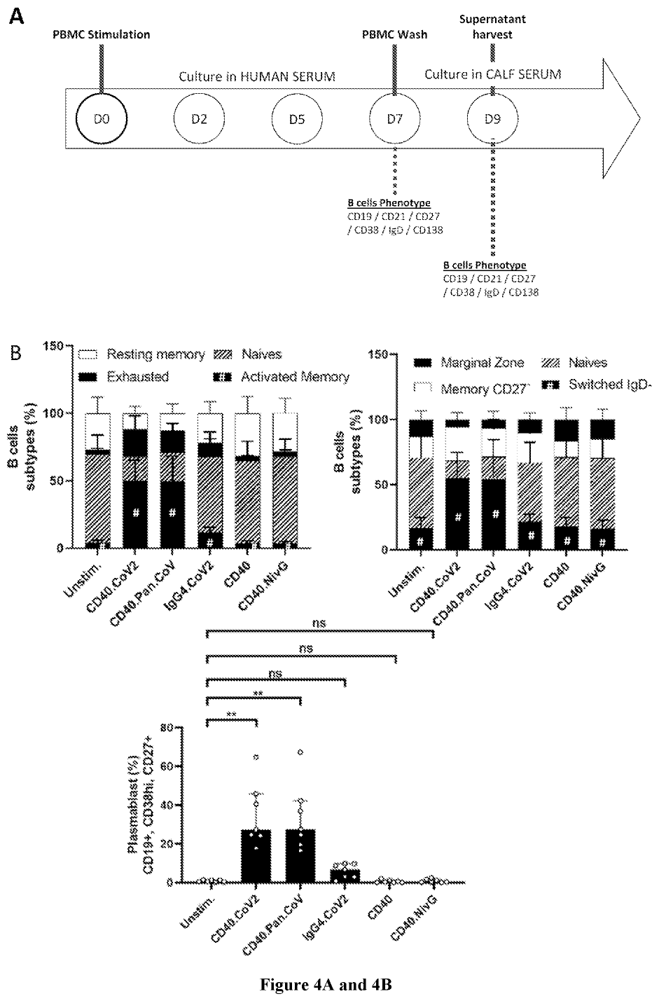

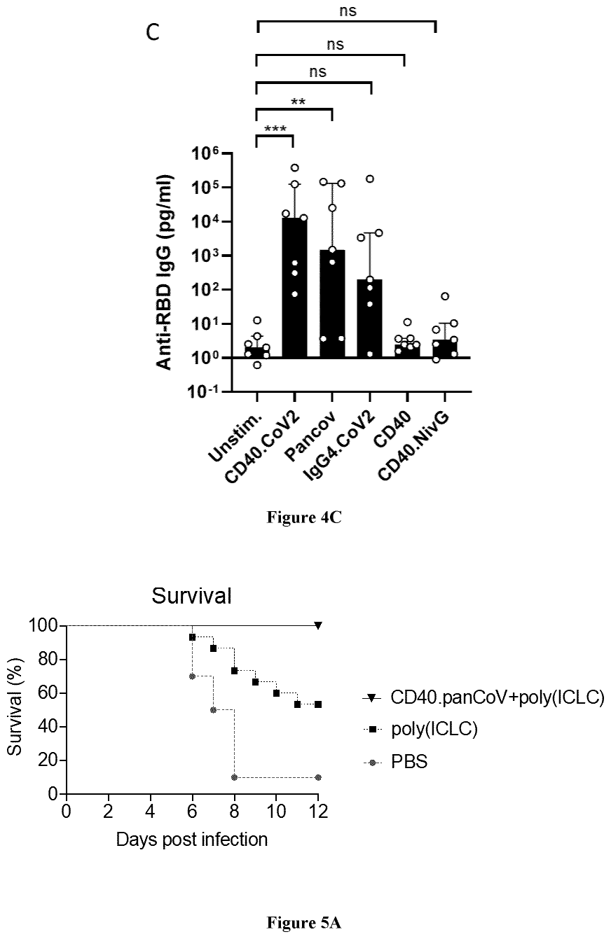

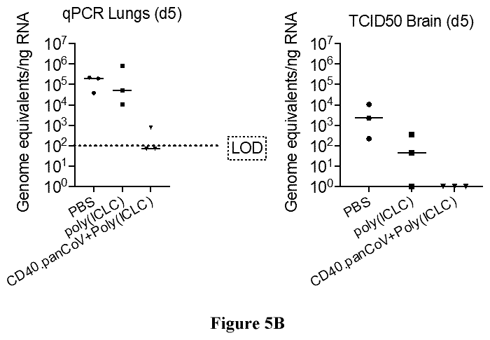

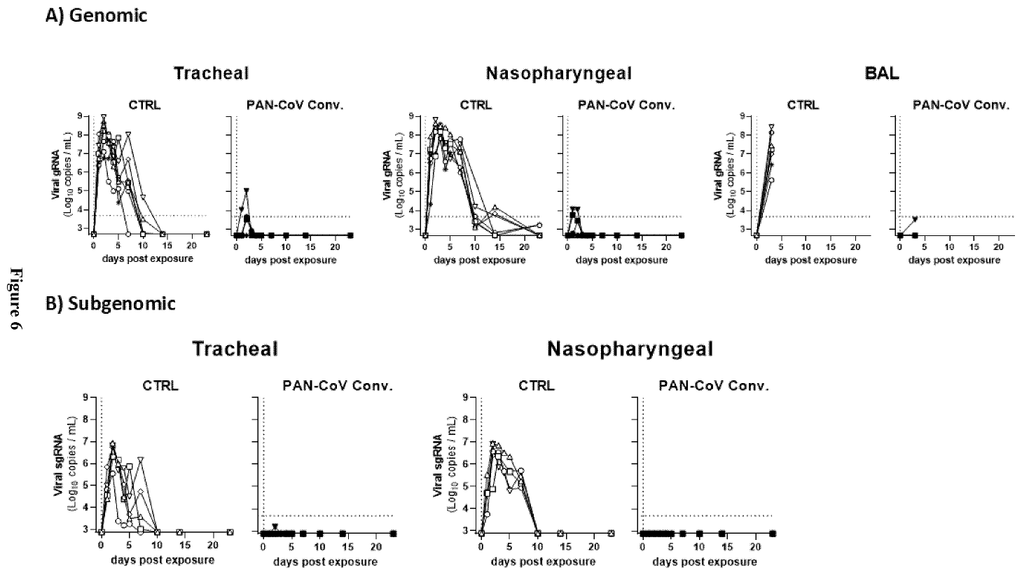

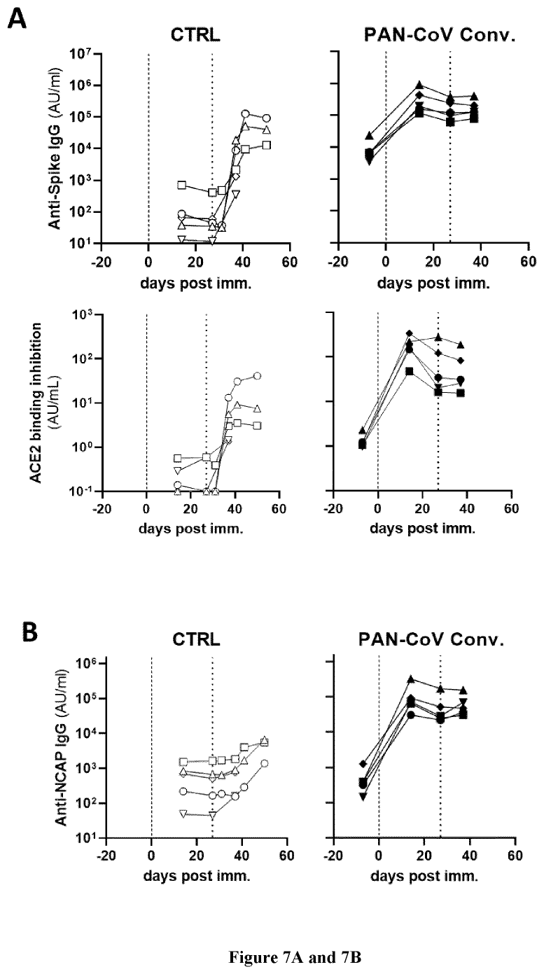

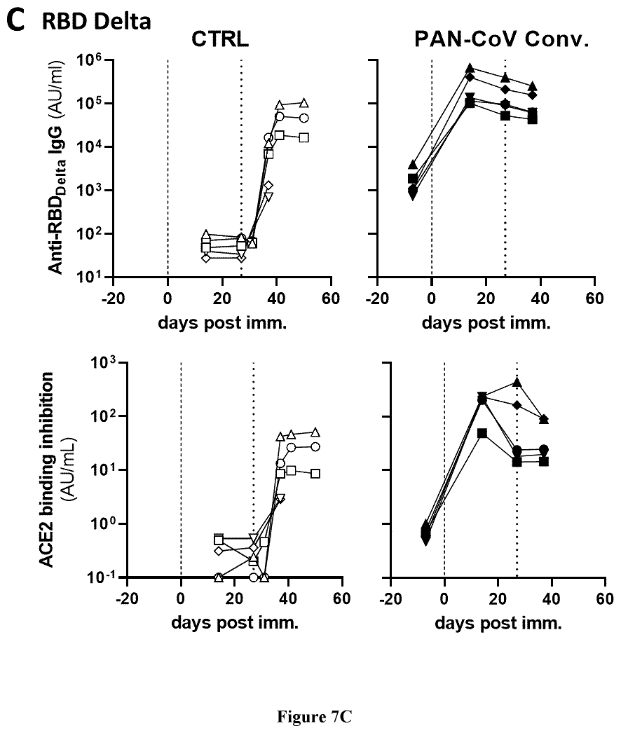

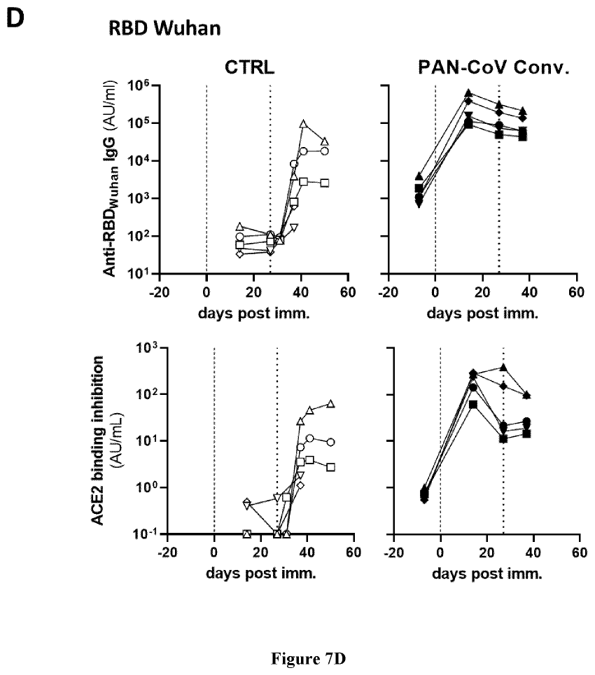

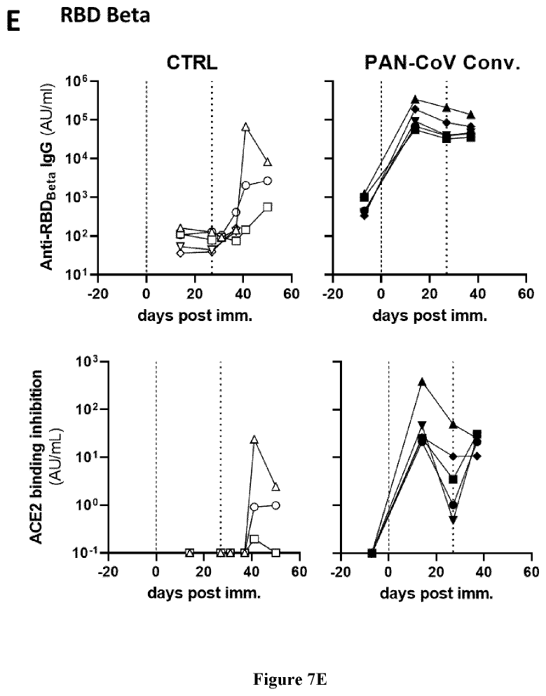

In particular, we generated the 2 constructs as depicted in FIG. 1. We confirm that the constructs binds to human CD40 (data not shown). The constructs were then tested via in vitro expansion of T cells in SARS-CoV-2-infected convalescent donor PBMC cultures with different overlapping peptide (OLP) pools specific for RBD and Npep2 regions of different Sarbecoviruses (FIG. 2). Since the homology between the RBD regions and the Npep2 regions is very high (FIG. 3), the constructs herein disclosed can be used as universal Sarbecovirus vaccines. In particular, the mean [min-max] percentage of homology between these 38 sarbecoviruses for Npep2 was 93.5 [89.7 to 100] % (FIG. 3). Beyond sequence homology across sarbecoviruses, we observed the vaccine T-cell epitopes to be highly conserved between SARS-CoV-2 and SARS-CoV-1 and the 32 sarbecoviruses, reaching 75 to 100% homology. More in-depth analysis showed that among all CD8+ T-cell epitopes, 62% (n=57) differed between SARS-CoV-2 and CoV-1 by at least one mutation, but these mutations did not affect HLA-Class I binding for a large majority of them (81%), as predicted by NetMHC4.. Moreover, two CD4+ T-cell epitopes included in the Npep2 (N301-315, and N306-320) and 6 CD8+ T-cell epitopes from Npep2 (N305-314; N306-315; N307-315; 308-317; 310-319; 311-319) were 100% homologous across all sarbecoviruses. Moreover, we observed very strong CD8+ T-cell response against Npep2 that is very important for long-term immunity (see FIG. 6c in Coléon, Séverin, et al. “Design, immunogenicity, and efficacy of a pan-sarbecovirus dendritic-cell targeting vaccine.” EBioMedicine 80 (2022): 104062.). Globally, these results confirm that Npep2 sequence is suitable for the design of a pan-sarbecovirus vaccine aiming to elicit broad cross-reactive T-cell responses. Following Pan-Sarbecovirus DC-targeting vaccine (CD40.Pan.CoV, i.e CD40.N2.RBDvSAD-Q vaccine) stimulation of SARS-CoV-2-infected convalescent donor PBMC cultures (FIG. 4A), we induced B cell activation and plasma cell differentiation (FIG. 4B) in vitro, able to produce anti-RBD IgG FIG. 4C). We demonstrated that K18 hACE2 tg×hCD40tg mice immunized twice with the CD40.Pan.CoV vaccine adjuvanted with poly-ICLC were protected from a lethal SARS-CoV-2 Wuhan challenge as compared to the controls animals (PBS or poly-ICLC) (FIG. 5A). Accordingly, the SARS-CoV-2 viral replication (genome equivalent/μg RNA) in the lungs and the viral infectious particles in the brain (PFU/mg of tissue) of the vaccinated mice were lower to undetectable than the controls (FIG. 5B). We next subcutaneously injected five convalescent cynomolgus macaques with 200 μg of CD40.Pan.CoV vaccine without adjuvant. Four weeks after immunization, the five CD40.Pan.CoV-immunized convalescent NHPs and six SARS-CoV-2-naïve animals (control group) were exposed to a total dose of 10e5 TCID₅₀ of SARS-CoV-2 B.1.617.2 variant virus (Delta strain). All naive animals became infected, as shown by the detection of viral genomic (gRNA, FIG. 6A) and sub-genomic (sgRNA, FIG. 6B) RNA in tracheal and nasopharyngeal swabs and broncho-alveolar lavages (BAL). We demonstrated that all convalescent cynomolgus macaques immunized with CD40.Pan.CoV following a challenge with Delta strain exhibited significantly lower viral genomic RNA levels (FIG. 6A) and viral subgenomic RNA levels (FIG. 6B) than the control animals. We also demonstrated that the immunization with the CD40.Pan.CoV was able to induce a binding and neutralizing Ab response against Spike (FIG. 7A), a binding Ab response against the Nucleocapside (FIG. 7B), as well as a cross-reactive binding and neutralizing Ab response against Wuhan, Delta and Beta RBD (FIG. 7C-E).

### REFERENCES

Throughout this application, various references describe the state of the art to which this invention pertains. The disclosures of these references are hereby incorporated by reference into the present disclosure.
1. Voysey, M. et al. Safety and efficacy of the ChAdOx1 nCoV-19 vaccine (AZD1222) against SARS-CoV-2: an interim analysis of four randomised controlled trials in Brazil, South Africa, and the UK. Lancet (London, England) 397, 99-111, (2020).
2. Corbett, K. S. et al. Evaluation of the mRNA-1273 vaccine against SARS-CoV-2 in nonhuman primates. N. Engl. J. Med 383, 1544-1555 (2020).
3. Polack, F. P. et al. Safety and efficacy of the BNT162b2 mRNA Covid-19 vaccine. N. Engl. J. Med. 383, 2603-2615 (2020).
4. Mercado, N. B. et al. Single-shot Ad26 vaccine protects against SARS-CoV-2 in rhesus macaques. Nature 586, 583-588 (2020).
5. Oliver, S. E. et al. The Advisory Committee on Immunization Practices' Interim Recommendation for Use of Pfizer-BioNTech COVID-19 Vaccine—United States, December 2020. MMWR Morb. Mortal. Wkly Rep. 69, 1922-1924 (2020).
6. Widge, A. T. et al. Durability of responses after SARS-CoV-2 mRNA-1273 Vaccination. N. Engl. J. Med. 384, 80-82 (2020).
7. Anderson, E. J. et al. Safety and immunogenicity of SARS-CoV-2 mRNA-1273 vaccine in older adults. N. Engl. J. Med. 383, 2427-2438 (2020).
8. Ella, R. et al. Safety and immunogenicity of an inactivated SARS-CoV-2 vaccine, BBV152: interim results from a double-blind, randomised, multicentre, phase 2 trial, and 3-month follow-up of a double-blind, randomised phase 1 trial. Lancet Infect Dis. 7, 950-961 (2021).
9. Levine-Tiefenbrun, M. et al. Initial report of decreased SARS-CoV-2 viral load after inoculation with the BNT162b2 vaccine. Nat. Med. 27, 790-782 (2021).
10. Logunov, D. Y. et al. Safety and efficacy of an rAd26 and rAd5 vector-based heterologous prime-boost COVID-19 vaccine: an interim analysis of a randomised controlled phase 3 trial in Russia. Lancet (Lond., Engl.) 397, 671-681 (2021).
11. Wu, Z. et al. Safety, tolerability, and immunogenicity of an inactivated SARS-CoV-2 vaccine (CoronaVac) in healthy adults aged 60 years and older: a randomised, double-blind, placebo-controlled, phase 1/2 clinical trial. Lancet Infect. Dis. 21, 803-812 (2021).
12. Marlin R,. et al. Targeting SARS-CoV-2 receptor-binding domain to cells expressing CD40 improves protection to infection in convalescent macaques. Nat Commun. 2021 Sep. 1; 12(1):5215. doi: 10.1038/s41467-021-25382-0. PMID: 34471122.

1.-18. (canceled)

19. An antibody comprising a heavy chain and a light chain, wherein the antibody is directed against a surface antigen of an antigen presenting cell and wherein:
the heavy chain is conjugated or fused to an Npep2 polypeptide that ranges from the residue at position 276 to the residue at position 411 in SEQ ID NO:1, and
the light chain is conjugated or fused to:
an RBD polypeptide that ranges from the amino acid residue at position 319 to the amino acid residue at position 541 in SEQ ID NO:2 and that comprises naturally occurring mutations K417N, L452R, T478K, E484K, and N501Y and that comprises non-naturally occurring mutation C538S or,
an RBD polypeptide that ranges from the amino acid residue at position 319 to the amino acid residue at position 541 in SEQ ID NO:2 and that comprises naturally occurring mutations K417N, L452R, T478K, E484Q, and N501Y and that comprises non-naturally occurring mutation C538S.

20. The antibody of claim 19 that is an IgG antibody.

21. The antibody of claim 20 that is an IgG4 antibody.

22. The antibody of claim 19 that is a chimeric antibody a humanized antibody.

23. The antibody of claim 22 that is a humanized antibody.

24. The antibody of claim 19 that is selected from an antibody that specifically binds to DC immunoreceptor (DCIR), MHC class I, MHC class II, CD1, CD2, CD3, CD4, CD8, CDI Ib, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fey receptor, LOX-1, and ASPGR.

25. The antibody of claim 19 that is specific for CD40.

26. The antibody of claim 25 that derives:
from the 12E12 antibody and comprises:
a heavy chain comprising the complementarity determining regions CDR1H, CDR2H, and CDR3H, the CDR1H having the amino acid sequence GFTFSDYYMY (SEQ ID NO:3), the CDR2H having the amino acid sequence YINSGGGSTYYPDTVKG (SEQ ID NO:4), and the CDR3H having the amino acid sequence RGLPFHAMDY (SEQ ID NO:5),
and a light chain comprising the complementarity determining regions CDR1L, CDR2L, and CDR3L, the CDR1L having the amino acid sequence SASQGISNYLN (SEQ ID NO:6) the CDR2L having the amino acid sequence YTSILHS (SEQ ID NO:7) and the CDR3L having the amino acid sequence QQFNKLPPT (SEQ ID NO:8).
or from the 11B6 antibody and comprises:
a heavy chain comprising the complementarity determining regions CDR1H, CDR2H, and CDR3H, the CDR1H having the amino acid sequence GYSFTGYYMH (SEQ ID NO:9), the CDR2H having the amino acid sequence RINPYNGATSYNQNFKD (SEQ ID NO:10), and the CDR3H having the amino acid sequence EDYVY (SEQ ID NO:11), and
a light chain comprising the complementarity determining regions CDR1L, CDR2L, and CDR3L, the CDR1L having the amino acid sequence RSSQSLVHSNGNTYLH (SEQ ID NO:12) the CDR2L having the amino acid sequence KVSNRFS (SEQ ID NO:13) and the CDR3L having the amino acid sequence SQSTHVPWT (SEQ ID NO:14).
or from the 12B4 antibody and comprises:
a heavy chain comprising the complementarity determining regions CDR1H, CDR2H, and CDR3H, the CDR1H having the amino acid sequence GYTFTDYVLH (SEQ ID NO:15), the CDR2H having the amino acid sequence YINPYNDGTKYNEKFKG (SEQ ID NO:16), and the CDR3H having the amino acid sequence GYPAYSGYAMDY (SEQ ID NO:17), and
a light chain comprising the complementarity determining regions CDR1L, CDR2L, and CDR3L, the CDR1L having the amino acid sequence RASQDISNYLN (SEQ ID NO:18) the CDR2L having the amino acid sequence YTSRLHS (SEQ ID NO:19) and the CDR3L having the amino acid sequence HHGNTLPWT (SEQ ID NO:20).

27. The antibody of claim 26 wherein the anti-CD40 antibody is selected from the group consisting of:
an antibody comprising a heavy chain variable region of sequence SEQ ID NO:21 and a light chain variable region of sequence SEQ ID NO:22;
an antibody comprising a heavy chain variable region of sequence SEQ ID NO:23 and a light chain variable region of sequence SEQ ID NO:22;
an antibody comprising a heavy chain variable region of sequence SEQ ID NO:24 and a light chain variable region of sequence SEQ ID NO:25;
an antibody comprising a heavy chain variable region of sequence SEQ ID NO:26 and a light chain variable region of sequence SEQ ID NO:27;
an antibody comprising a heavy chain variable region of sequence SEQ ID NO:28 and a light chain variable region of sequence SEQ ID NO:29; and
an antibody comprising a heavy chain variable region of sequence SEQ ID NO:30 and a light chain variable region of sequence SEQ ID NO:31.

28. The antibody of claim 19 that is specific for Langerin.

29. The antibody of claim 28 that comprises:
a heavy chain comprising the complementarity determining regions CDR1H, CDR2H, and CDR3H of the 15B10 antibody and a light chain comprising the complementarity determining regions CDR1L, CDR2L, and CDR3L of the 15B10 antibody, or
a heavy chain comprising the complementarity determining regions CDR1H, CDR2H, and CDR3H of the 2G3 antibody and a light chain comprising the complementarity determining regions CDR1L, CDR2L, and CDR3L of the 2G3 antibody, or
a heavy chain comprising the complementarity determining regions CDR1H, CDR2H, and CDR3H of the 4C7 antibody and a light chain comprising the complementarity determining regions CDR1L, CDR2L, and CDR3L of the 4C7 antibody.

30. The antibody of claim 29 that is selected from the group consisting of:
an antibody comprising a heavy chain variable region of sequence SEQ ID NO:32 and a light chain variable region of sequence SEQ ID NO:33;
an antibody comprising a heavy chain variable region of sequence SEQ ID NO:34 and a light chain variable region of sequence SEQ ID NO:35; and
an antibody comprising a heavy chain variable region of sequence SEQ ID NO:36 and a light chain variable region of sequence SEQ ID NO:37.

31. The antibody of claim 19 wherein the heavy chain and/or the light chain is fused to the RBD polypeptide or Npep2 polypeptide via the linker selected from the group consisting of SEQ ID NO:38 (FlexV1), SEQ ID NO:39 (f1), SEQ ID NO:40 (f2), SEQ ID NO:41 (f3), and SEQ ID NO:42 (f4).

32. The antibody of claim 19 that comprises (i) a light chain that is fused to the RBD polypeptide to form the fusion protein as set forth in SEQ ID NO:43 and (ii) the heavy chain that is fused to the Npep2 polypeptide to form the fusion protein as set forth in SEQ ID NO:45.

33. The antibody of claim 19 that comprises (i) a light chain that is fused to the RBD polypeptide to form the fusion protein as set forth in SEQ ID NO:44 and (ii) the heavy chain that is fused to the Npep2 polypeptide to form the fusion protein as set forth in SEQ ID NO:45.

34. A nucleic acid that encodes the heavy chain and/or the light chain of the antibody of claim 19.

35. A vector comprising the nucleic acid of claim 34.

36. A host cell which has been transfected, infected, or transformed by the nucleic acid of claim 34 and/or a vector comprising the nucleic acid of claim 34.

37. A vaccine composition comprising the antibody of claim 19.

38. A method for vaccinating a subject in need thereof against Sarbecoviruses comprising administering a therapeutically effective amount of the antibody of claim 19.
